SEQUENCE LISTING (2 sequences)

SEQ ID NO: 1 - protein, 4 aa, Artificial
  Synthetic Peptide
WMDF

SEQ ID NO: 2 - protein, 5 aa, Artificial
  Synthetic Peptide
AWMDF

(19) United States
(12) Patent Application Publication
(10) Pub. No.: US 2006/0135406 A1
(43) Pub. Date: Jun. 22, 2006

(54) Compositions and methods for treating pathologies that necessitate suppression of gastric acid secretion

(76) Inventors: Sabina Glozman, Rehovot (IL); Ayelet David, Negev (IL); Lada Paul, Vancouver (CA)

Correspondence Address: FENNEMORE CRAIG, 3003 NORTH CENTRAL AVENUE, SUITE 2600, PHOENIX, AZ, 85012, US

(21) Appl. No.: 11/351,001

(22) Filed: Feb. 9, 2006

Related U.S. Application Data
(63) Continuation-in-part of application No. 10/682,937, filed on Oct. 14, 2003.
(63) Continuation-in-part of application No. PCT/IB04/02745, filed on Aug. 25, 2004.
(60) Provisional application No. 60/497,930, filed on Aug. 27, 2003.
(60) Provisional application No. 60/544,318, filed on Feb. 17, 2004.
(60) Provisional application No. 60/655,471, filed on Feb. 23, 2005.
(60) Provisional application No. 60/682,808, filed on May 20, 2005.

(30) Foreign Application Priority Data
Oct. 14, 2002 (IL) 152289

(51) Int. Cl. A61K 38/54; A61K 31/4439

(52) U.S. Cl. 514/2; 514/338

(57) ABSTRACT

The present invention is related to novel oral compositions comprising an irreversible gastric H⁺/K⁺-ATPase proton pump inhibitor (PPI) as a gastric acid secretion inhibitor, pentagastrin (PG) or a PG analogue as an activator of parietal cells in the gastric lumen. In a preferred embodiment, the composition further comprises at least one agent that preserves the availability of PG in the gastric fluids, thus enabling PG to act locally in the stomach. Unexpectedly, the compositions of the present invention exhibit anti-acid activity locally in the stomach that is meal-independent, exhibit fast onset and prolonged inhibition of acid secretion.

### CROSS-REFERENCE TO RELATED APPLICATIONS

This application is a continuation-in-part of U.S. application Ser. No. 10/682,937 filed on Oct. 14, 2003, and a continuation-in-part of International application PCT/IB2004/002745 filed Aug. 25, 2004, which claims the benefit of U.S. Provisional Application No. 60/497,930 filed Aug. 27, 2003 and U.S. Provisional Application No. 60/544,318 filed Feb. 17, 2004; this application further claims the benefit of U.S. Provisional Application No. 60/655,471 filed Feb. 23, 2005 and U.S. Provisional Application No. 60/682,808 filed May 20, 2005, the content of each the above cited applications of which is expressly incorporated herein by reference thereto.

### FIELD OF THE INVENTION

The present invention relates to novel oral compositions for inhibition of gastric acid secretion that possess fast onset, prolonged inhibition effect on gastric acid secretion and are meal-independent.

### BACKGROUND OF THE INVENTION

A wide number of pathological conditions are characterized by the need to suppress gastric acid secretion. Such conditions include, but are not limited to Zollinger/Ellison syndrome (ZES), gastroesophageal reflux disease (GERD), peptic ulcer disease, duodenal ulcers, esophagitis, and the like. Conditions such as peptic ulcers can have serious complications and represent some of the most prevalent diseases in industrialized nations.

Presently, the main therapies employed in the treatment of GERD and peptic ulcer diseases include agents for reducing the stomach acidity, for example by using the histamine H₂-receptor antagonists or proton pump inhibitors (PPI's). PPI's act by inhibiting the parietal cell H⁺/K⁺ ATPase proton pump responsible for acid secretion from these cells. PPI's, such as, omeprazole, and its pharmaceutically acceptable salts are disclosed for example in EP 05129, EP 124495 and U.S. Pat. No. 4,255,431.

PPI agents are acid-labile pro-drugs that are usually administered in enteric-coated granules. Following their absorption in the small intestine PPIs, which are weak bases, preferentially accumulate within the acid milieu of parietal cells. The acid environment within the acid milieu of parietal cells causes the conversion of the pro-drugs into the active sulfenamids, which are the active agents that bind and inhibit the parietal cell H⁺/K⁺ ATPase pumps.

Despite their well-documented efficacy, PPIs have notable limitations. The time of dosing and ingestion of meals may influence the pharmacokinetics of these agents as well as their ability to suppress gastric acid secretion (Hatlebakk et al., Aliment Pharmacol Ther. 2000; 14(10):1267-72). Specifically, the PPI must be taken prior to ingestion of food in order to achieve optimal suppression of gastric acid secretion. Furthermore, PPIs have a relatively slow onset of pharmacological action and may require several days to achieve maximum acid suppression and symptom relief, limiting their usefulness in on-demand GERD therapy (Sachs G, Eur J Gastroenterol Hepatol. 2001;13 Suppl 1:S35-41). Moreover, PPIs fail to provide 24-h suppression of gastric acid and nocturnal acid breakthrough that leads to heartburn pain in GERD patients and occurs even with twice-daily dosing of PPIs (Tytgat G N, Eur J Gastroenterol Hepatol. 2001;13 Suppl 1:S29-33). Finally, these drugs exhibit substantial inter-patient variability in pharmacokinetics and may have significant interactions with other drugs (Hatlebakk et al., Clin Pharmacokinet. 1996; 31(5):386-406). Thus, an improvement of PPI-mediated activity is a well-recognized challenge in gastroenterology.

Pentagastrin (PG) (β-alanyl-L-tryptophyl-L-methionyl-L-aspartyl-L-phenyl-alanyl amide; SEQ ID NO:2) is a pentapeptide containing the carboxyl terminal tetrapeptide of gastrin. This carboxyl terminal tetrapeptide is the active portion found in essentially all natural gastrins. In animals, PG acts to induce gastric acid secretion mainly via induction of histamine release from enterochromafin-like (ECL) cells residing in the stomach. The release of histamine and the consequent activation of histamine receptors residing on the parietal cells, leads to the activation of the parietal cells to actively secrete proton ions to the gastric lumen. It is also possible that PG acts directly on the parietal cells to induce its activation. PG is typically used in the art as a diagnostic agent for the evaluation of gastric acid secretory function.

The low solubility of PG in acidic environment and the fact that PG is prone to pepsin degradation in the stomach, rendered its use as an inducer of gastric acid secretion following oral administration clearly unexpected until Applicants discovery. Prior to Applicants discovery, PG was considered by anyone skilled in the art to only be effectively active at inducing acid secretion if administered via parenteral routes. Indeed, no effect on acid secretion was noted in four normal subjects subjected to oral administration of PG, whereas some effect was noted in three additional patients with gastrointestinal abnormalities (Morrell & Keynes Lancet. 1975; 2(7937):712). In fact, this study was cited in a pharmacology textbook as a proof of lack of PG activity when administered orally (Martindale Thirty-second edition, p1616, the Chapter: “Supplementary Drugs and Other Substances”).

WO01/22985 to Pisegna et al. (the '985 publication) discloses the use of PG administered by injection in conjunction with a proton pump inhibitor (PPI). According to the '985 publication, administration of PG in combination with a PPI increases the efficacy of the PPI in reducing/mitigating excess gastric acid secretion. The '985 publication discloses and teaches that PG should preferably be administered by injection (e.g., subcutaneous injection). The '985 publication neither suggest that PG may be active locally in the stomach, nor discloses the use of PG preservation agents to preserve the biological activity of PG activity in the stomach in order to achieve local effect in the gastric lumen.

De Graef et al., Gastroenterology, 91, 333-337 (1986) (De Graef publication) discloses that omeprazole is more effective in inhibiting gastric acid secretion when administered to dogs pretreated intravenously with PG. There is no mention in the De Graef publication that oral administration of PG would be effective by acting locally in the gastric lumen to potentiate the effect of omeprazole.

U.S. Pat. Nos. 6,489,346; 6,645,988; and 6,699,885; to Phillips jointly the “Phillips patents”) disclose pharmaceutical compositions and methods of treating acid-caused gastrointestinal disorders using oral compositions consisting of a PPI, at least one buffering agent and specific parietal cell activators. The parietal cell activators disclosed in the Phillips patents include, for example, chocolate, sodium bicarbonate, calcium, peppermint oil, spearmint oil, coffee, tea and colas, caffeine, theophylline, theobromine and amino acids residues. As indicated in the Phillips patents, all these proposed parietal cell activators induce the release of endogenous gastrin that exerts both inhibitory and stimulatory effects on acid secretion by activating both CCK-A and CCK-B receptors. The Phillips patents do not disclose or suggest the use of activators such as pentagastrin which possess a solely stimulatory activity by binding only to CCK-B receptors.

The development of an effective treatment for pathologies in which inhibition of gastric acid secretion is required would fulfill a long felt need. Despite the wide-spread use of PPI's, a need still exist for increasing the PPI efficacy, e.g., faster effective onset, prolonged effect including night time acid breakthrough, greater effect at reduced dosage and meal-independent administration.

### SUMMARY OF THE INVENTION

It is the object of the present invention to provide oral compositions for inhibition of gastric acid secretion that are meal-independent and exhibit fast onset with prolonged inhibition effect on gastric acid secretion.

It is another object of the present invention to provide oral compositions for inhibition of gastric acid secretion comprising an irreversible gastric H⁺/K⁺-ATPase proton pump inhibitor (PPI) and a parietal cell activator, wherein the PPI anti-acid activity is meal-independent and exhibit fast onset and prolonged inhibitory effect on acid secretion.

In one embodiment, the present invention relates to oral compositions comprises an irreversible gastric H⁺/K⁺-ATPase proton pump inhibitor (PPI) as a gastric acid secretion inhibitor, pentagastrin (PG) and/or a PG analogue as an activator of parietal cells and one or more agents that preserve the availability of PG in the gastric fluids, so that the biological activity of PG is maintained thus enabling PG to act locally in the stomach. Unexpectedly, the compositions of the present invention possess anti-acid activity in the stomach that is meal-independent and exhibit fast onset and prolonged inhibition of acid secretion. The present compositions may be used for treating a subject suffering from chronic or acute disorders in which suppression of acid secretion in the stomach is required.

The proton pump inhibitors (PPIs) according to the present invention are compounds that inhibit the activity of the H⁺/K⁺-adenosine triphosphatase (ATPase) proton pump in the gastric parietal cells. In its pro-drug form, PPI is non-ionized and therefore is capable of passing through the cellular membrane of the parietal cells. Once reaching the parietal cells, the non-ionized PPI moves into the acid-secreting portion of activated parietal cells, the secretory canaliculus. The PPI trapped in the canaliculus becomes protonated, thus converted to the active sulfenamide form that can form disulfide covalent bonds with cysteine residues in the alpha subunit of the proton pump, thereby irreversibly inhibiting the proton pump.

As mentioned above, the present invention is based on the inventors surprising discovery that PG is active locally in the stomach when administered orally, preferably by acting locally in the gastric lumen to activate the parietal cells. Active parietal cells have an acidic pH, which is required for the conversion of the PPI to the active protonated sulfenamide form. Therefore, the synchronized activation of the parietal cells by PG acting directly in the gastric lumen maximizes the inhibition of the pumps by the PPI.

The oral compositions of the present invention exhibit the following advantages over the known PPI-based compositions aimed to reduce gastric acid secretion. The present compositions permit activation of the parietal cells by PG without any side effects associated with systemic administration of PG due to the local effect of PG in the gastric lumen. Pre-activation of parietal cells by PG facilitates the conversion of the PPI to the active sulfenamide form leading to fast onset of the effect of PPI. Furthermore, the present compositions exhibit fast onset of anti-acid activity in the stomach in a meal-independent manner. Thus, the combined active agents in the oral compositions provide an efficient solution for acute conditions in which fast reduction of acid secretion is required. Finally, the present oral compositions provide prolonged suppression of gastric acid secretion for at least 24 h using a single medication.

The oral compositions according to the present invention comprise PG or a PG analogue as a local activator of parietal cells in the gastric lumen. In addition to PG that comprises the amino acid sequence βAla-Trp-Met-Asp-PheNH₂ (SEQ ID NO:2), this invention contemplates the use of gastrin or PG analogues or derivatives thereof as parietal cell activators. Such variants include, but are not limited to the 34-, 17-, and 14-amino acid species of gastrin, and other truncation variants comprising the active C-terminal tetrapeptide of gastrin Trp-Met-Asp-PheNH₂ (SEQ ID NO:1), which is reported in the literature to have full pharmacological activity (see Tracey and Gregory (1964) Nature (London), 204: 935).

Also included are variants of gastrin and/or truncated gastrins where native amino acids are replaced with conservative substitutions. Various analogues of these molecules are also included, for example, but not limited to the N-protected derivative of PG Boc-βAla-Trp-Met-Asp-PheNH₂ in which Boc is tert-butyloxycarbonyl group or F-Moc-βAla-Trp-Met-Asp-PheNH₂ in which Moc is methoxycarbonyl.

The oral compositions according to the present invention may further comprise one or more gastric acid stimulants in combination with PG or a PG analogue in order to obtain better activation of parietal cells and synchronization between the activation of parietal cells and the absorption of PPI in blood in a meal-independent manner. The term “gastric acid stimulant” refers to any agent that is capable of stimulating gastric acid secretion via direct or indirect effect on parietal cells. Preferred gastric acid stimulants to be used in combination with PG or a PG analogue are small dicarboxylic and tricarboxylic acids such as succinic acid, maleic acid, citric acid and fumaric acid, or the salt thereof. The dicarboxylic or tricarboxylic acids are present in a pharmacological effective amount to stimulate acid secretion.

In a non-limiting embodiment, the oral compositions further comprise one or more agents that preserve the availability of PG in the acidic gastric fluids. The preservation agents preferably are in an amount sufficient to preserve the availability of PG in the gastric fluids by retaining the solubility of PG in the gastric fluids and/or preventing its degradation, so that the local biological activity of PG in the stomach is preserved and activates the parietal cells.

Preferred preservation agents include, for example, pH regulating agents and pepsin inhibitors. Preferably when the pH regulating agent is dissolved in the gastric juice it is capable of temporally elevating the pH of the gastric fluids to a value in which pepsin is inhibited, thereby inhibiting pepsin degradation of PG in the gastric fluids. Since PG is soluble only in alkaline conditions, the temporary elevation of the pH in the gastric fluids ensures that at least significant proportion of PG remains soluble in the gastric fluids.

It is noted that any weak or strong base (and mixtures thereof) can be utilized as the pH regulating agent in the present oral compositions. The pH regulating agent is present in the composition in an amount sufficient to substantially preserve the stability and the solubility of PG in the acidic gastric fluids. Therefore, the pH regulating agent, when dissolved in the gastric juice, is capable of elevating the pH of the stomach to a value sufficient to achieve adequate availability of PG to effect therapeutic action.

According to a preferred embodiment, the pH regulating agent is in an amount sufficient to elevate the pH of the gastric fluids to a value of at least 4, 4.5, or 4.8. more preferably at least 5, 5.5 or 5.8, and. preferably at least 6.0. In one embodiment, the pH of the gastric fluid is elevated for a time period sufficient for PG to reach and activate the parietal cells locally in the stomach. Preferably, the pH regulating agent is in amount sufficient to elevate the pH of the gastric fluids for at least 10 to 20 minutes, more preferably for at least 20 to 30 minutes, and most preferably for at least 30 to 60 minutes.

In one embodiment, the pH regulating agent is in a sustained formulation to maintain the elevated pH.

In preferred embodiment, the pH regulating agent is capable of elevating the pH of the gastric fluids to a value above 5 for a time period ranging from 5 to 60 minutes, preferably for a time period ranging from 5 to 30 minutes. Thus, the pH regulating agent according to the present invention preserves the solubility of PG in the gastric fluids for a time period sufficient for PG to activate the parietal cells. Furthermore, the temporal alkali condition in the gastric fluid prevents the degradation of PG by pepsin that is active only in acidic pH.

The present invention further relates to a kit for treatment of gastric-acid secretion-related disorders based on PPI, PG and/or a PG analogue and adjustable amounts of pH regulating agents. The kit comprises two different separate doses: an initial dose for the early stage of treatment containing an effective amount of PPI granules, PG and/or a PG analogue combined with high amount of pH regulating agents and a continuance dose containing comparable amount of PPI granules and PG and/or a PG analogue combined with low amount of pH regulating agents. The inventors unexpectedly found that the amount of pH regulating agents required in order to preserve the stability of PG in the gastric fluids may be lowered during the continuance stage of treatment. This is based on the observation that basic pH in the stomach is established during the initial stage of PPI treatment.

According to specific embodiments, the initial dose contains pH regulating agents in an amount sufficient to raise the acidic pH of the stomach to a pH in which the stability of PG is maintained, preferable to a pH above about 5.5-6.0. The continuance dose containing low amount of pH regulating agents and should be taken only following the ingestion of PPIs for several days and the consequent establishment of more basic pH in the stomach. The amount of pH regulating agents in the continuance dose is sufficient to elevate the pH of the stomach from about pH 4.0-5.0 (the pH in stomach during chronic treatment of PPI) to a pH above about pH 6.0 (the pH sufficient to preserve the stability of PG in the stomach).

In a specific embodiment, the initial dose of the kit contains one or more pH regulating agents in an amount sufficient to raise the pH in the stomach to a pH of at least 4.5, and the continuance dose contains one or more pH regulating agents in an amount less than in the initial dose, but sufficient to raise the pH in the stomach to a pH of at least 4.5.

In a preferred embodiment, both initial and continuance doses comprise one or more pH regulating agents in an amount sufficient to raise the pH in the stomach to a pH of at least 5.5, more preferably to a pH of at least 6.0.

In another preferred embodiment, the initial dose comprises one or more pH regulating agents in an amount sufficient to raise the pH in the stomach by at least 2.5 to 5.5 pH units and the pH regulating agent in the continuance dose is in an amount sufficient to raise the pH in the stomach by at least 1 to 3 pH units.

In more preferred embodiment, the initial dose comprises one or more pH regulating agents in an amount sufficient to raise the pH in the stomach by at least 4 to 5 pH units and the pH regulating agent in the continuance dose is in an amount sufficient to raise the pH in the stomach by at least 1.5 to 2.5 pH units.

In another embodiment, the kit of the present invention comprises at least two continuance doses, a first and second continuance dose, wherein the amount of pH regulating agent in the second continuance dose is less than the amount of pH regulating agent in the first continuance dose.

The kit of the present invention exhibits a significant advantage since the adjustable amount of pH regulating agents prevents alkalosis following the chronic ingestion of high amount of buffer. High dose of buffer such sodium bicarbonate is not recommended especially in patients on sodium-restricted diet. Furthermore, high dose of sodium bicarbonate is contradicted in patients with metabolic alkalosis and hypocalcemia, and should be used with caution in patients with hypokalemia and respiratory alkalosis. Thus, the combination kit of the present invention provides an effective solution for patients that would like to use immediate-release PPI-based compositions chronically but are instructed not to ingest high amount of buffer.

The PPI in the kit of the present invention is formulated either as non-enteric-coated or enteric-coated PPI granules. Both will provide an immediate release profile and fast absorption of PPI in blood due to the basic environment of the stomach. The basic environment rapidly induces the dissolving of the enteric-coating from the PPI granules, thereby permitting immediate release of the PPI from the granules and fast absorption in blood.

According to various embodiments, the present compositions further comprise other agents that preserve the availability of PG in the acidic gastric fluids. Such agents are for example pepsin inhibitors (i.e., pepstain and its derivative bacitracin—cyclic dodecapeptide) that reduce the degradation of the peptide in the stomach or mucolytic agents that reduce the viscosity of the gastric mucosa, thereby accelerating the ability of PG to reach the cells responsible for acid secretion. Such mucolytic agents are for example reducing agents such as N-acetyl cysteine, dithiothreitol, citric acid or mannitol. The present compositions may further comprise an antibiotic effective against bacteria residing in the stomach.

The active ingredients of the present invention may be formulated in a single oral dosage form, preferably a solid dosage form. Liquid dosage forms such as suspensions may be used as well. Thus, in one embodiment the PPI, PG and the agent that preserves the availability of PG in the gastric fluids may be formulated as multi-layered tablets, suspension tablets, effervescent tablets, chewable tablets, powder for suspension, pellets, granules, hard gelatin capsules comprising multiple beads, or soft gelatin capsules containing a lipid-based vehicle.

According to one embodiment, the solid dosage form of the present invention is a capsule or a multi-layered tablet containing PPI particles coated with either enteric pH-dependent release polymers or non-enteric time-dependent release polymers, particles of PG and particles of one or more pH regulating agents. In order to ensure that the activation of parietal cells in the gastric lumen by PG is synchronized with the absorption of the PPI in the proximal part of the small intestine, the single oral dosage form may comprise PG beads coated with time-dependent release polymer that extends the PG releasing time in the stomach. Thus, the extension of PG release in the stomach permits the synchronization between the activity of PG and the activity of the PPI on the parietal cells.

The active ingredients of the present invention may also be formulated in separate dosage forms. For example, PG and the agent that preserves the availability of PG in the gastric fluids may be formulated in an oral suspension or a solid dosage form such as capsules, tablets, suspension tablets, or effervescent tablets and the PPI may be formulated in a separate solid dosage form, preferably capsules or tablets comprising beads with enteric pH-dependent release polymers or non-enteric time-dependent release polymers. The separate dosage forms may be provided as a kit containing PG and the agent that preserves the availability of PG in the gastric fluids in one dosage form and the PPI in a separate dosage form. In this case, the PG is administered in conjunction with the PPI so that there is at least some chronological overlap in their physiological activity. The PPI and PG can be administered simultaneously and/or sequentially.

The PPI particles used in the present invention may be coated with either enteric pH-dependent release polymer, non-enteric time-dependent release polymer or may be without coating layer. The stability of the non-coated PPI while passing the stomach is preserved by the one or more pH regulating agents present in the composition. It was previously demonstrated that the absorption of buffered suspension of non-enteric-coated PPI in the proximal part of the small intestine is faster than the absorption of the enteric-coated PPI granules (Pilbrant and Cederberg, Scand. J. Gastroenterol 1985:20 (supp. 108): 113-120). Therefore, it is not necessary to delay the release of PG in the stomach if non-coated PPI particles are used in the composition. However, when coated PPI particles are used, it is required to synchronize the release of the PPI with the release of PG by delaying the release of PG in the stomach for example by using polymeric coated PG particles.

In another embodiment, the present invention is directed to a method of treating a subject suffering from a disorder in which suppression of gastric acid secretion is required or a disorder normally treated by suppression of gastric acid secretion. The method comprising administering to the subject a pharmaceutical composition comprising a PPI as a gastric acid secretion inhibitor, PG or a PG analogue as an activator of parietal cells in the gastric lumen, and at least one preservation agent in an amount sufficient to preserve the availability of PG in the gastric lumen.

The compositions of the present invention may be used for preventing or treating pathologies in a mammal in which inhibition of gastric acid secretion is required. Preferably the mammal is human. The compositions of the present invention are effective both in treating the pathologies and in minimizing the risk of development of such pathologies before onset of symptoms.

The pharmaceutical compositions of the present invention may be used in a wide number of pathological conditions that are treated by suppression of gastric acid secretion. Such conditions include, but are not limited to Zollinger/Ellison syndrome (ZES), gastroesophageal reflux disease (GERD), esophagitis, peptic ulcer diseases, duodenal ulcers, gastritis and gastric erosions, dyspepsia, and the like.

The present invention also includes an oral pharmaceutical kit. The kit typically comprises as active ingredients a pharmaceutically effective amount of: (i) a peptide comprising the amino acid sequence of SEQ ID NO:1; (ii) an irreversible gastric H⁺/K⁺-ATPase proton pump inhibitor (PPI); and (iii) at least one agent that preserves the availability of the peptide in the gastric fluids. In one embodiment, the active ingredients are formulated in separate dosage unit forms. The kit may be used to treat or prevent a disorder in a subject in which suppression of gastric acid secretion is required by administering to a subject the active ingredients. The peptide is typically administered simultaneously, prior to or following the administration of the PPI.

These and further embodiments will be apparent from the detailed description and examples that follow.

### BRIEF DESCRIPTION OF THE FIGURES

FIG. 1 demonstrates that NaHCO₃ preserves PG stability in artificial gastric fluid;

FIG. 2 demonstrates the percentage of non-degraded PG in various pH values;

FIG. 3 is a schematic illustration of a double-layered tablet comprising PG, non-enteric-coated omeprazole and a pH regulating agents;

FIG. 4 is a schematic illustration of PG granules used in the multi particulate capsule formulation;

FIG. 5 is a schematic illustration of a capsule comprising time release-coated beads;

FIG. 6 demonstrates that PG stimulates gastric acid secretion in rats in a dose-dependent manner;

FIG. 7 demonstrates that PG enhances PPI-mediated effect on gastric acid secretion in rats;

FIG. 8 demonstrates that Lansoprazole inhibits gastric acid secretion in conscious animals in a dose-dependent manner;

FIG. 9 demonstrates that PG increases the efficacy of Lansoprazole in the blockade of gastric acid secretion when Lansoprazole is administered prior to PG (A) and not when Lansoprazole is administered following PG (B);

FIG. 10 demonstrates that administration of Lansoprazole in combination with PG during 3 consecutive days resulted in significantly higher intragastric pH (A) and lower gastric acid secretion (B) as compared to Lansoprazole alone.

FIG. 11 details the amount of sodium bicarbonate required in order to reach a pH above 6 in gastric stimulated fluid.

### DETAILED DESCRIPTION OF THE INVENTION

The term “preservation agent” as used herein, includes any agent that preserves the availability of PG in gastric fluids by retaining the solubility of PG in gastric fluids and/or by preventing PG's degradation. Preferably the preservation agent preserves at least a substantial amount of PG in a soluble and non-degraded form in the gastric juice, so that the biological activity of PG in the stomach is maintained. In a preferred embodiment, the preservation agent preserves at least 50%, more preferably at least 60%, and still more preferably at least 70% of the PG administered for a time sufficient to activate the parietal cells.

Preferred preservation agents include, for example, pH regulating agents and pepsin inhibitors. The term “pH regulating agent” as used herein, includes any agent used to regulate the pH of the stomach, preferably any pharmaceutically appropriate weak base or strong base (and mixtures thereof) that, when formulated or delivered with (e.g., before, during and/or after) the peptide comprising SEQ ID NO:1, (preferably PG), functions to temporally elevate the pH in the gastric lumen to a value that substantially preserves the availability of PG in the stomach. In a preferred embodiment, the pH regulating agent retains the solubility and/or inhibits the acid degradation of PG for a sufficient time that PG acts local to activate parietal cells located in the gastric lumen.

The term “biological activity of PG in the stomach” refers to its activation of parietal cells located in the gastric lumen.

The term “in conjunction with” means that when the PPI and the PG are administered in separate dosage forms, there is at least some chronological overlap in their physiological activity. Thus the PPI and PG can be administered simultaneously and/or sequentially.

The present invention is based on the surprising discovery that PG is capable of remaining active following oral administration to activate the parietal cells, preferably by acting locally in the stomach. Importantly, parietal cell activation is required for the conversion of the PPI pro-drug to the active form that acts as an irreversible inhibitor of the gastric H⁺/K⁺-ATPase proton pump. The oral compositions of the present invention provide a unique combination of active agents that increase the efficacy of the PPI in inhibiting gastric acid secretion.

The compositions of the present invention may be used for preventing or treating pathologies in a mammal in which inhibition of gastric acid secretion is required. The compositions of the present invention are effective both in treating the pathologies and in minimizing the risk of development of such pathologies before onset. Such pathologies include for example: reflux esophagitis, gastritis, duodenitis, gastric ulcer and duodenal ulcer. Furthermore, the compositions of the present invention may be used for treatment or prevention of other gastrointestinal disorders where gastric acid inhibitory effect is desirable, e.g. in patients on nonsteroidal anti-inflammatory drugs (NSAID) therapy (including low dose aspirin), in patients with Non Ulcer Dyspepsia, in patients with symptomatic gastro-esophageal reflux disease (GERD), and in patients with gastrinomas. They may also be used in patients in intensive care situations, in patients with acute upper gastrointestinal bleeding, in conditions of pre-and postoperatively to prevent aspiration of gastric acid and to prevent and treat stress ulceration. Further, they may be useful in the treatment of Helicobacter infections and diseases related to these. Other conditions well suited for treatment include, but are not limited to Zollinger-Ellison syndrome (ZES), Werner's syndrome, and systemic mastocytosis.

The parietal cell activator according to the present invention is preferably PG having the amino acid sequence denoted as SEQ ID NO:2. However, any PG analog that comprises the C-terminal tetrapeptide of gastrin Trp-Met-Asp-PheNH₂ (denoted as SEQ ID NO:1) may be used as a parietal cell activator. Such analogues include, but are not limited to the 34-, 17-, and 14-amino acid species of gastrin, and other truncation variants. Also included are variants of gastrin and/or truncated gastrins where native amino acids are replaced with conservative substitutions. Also included are various analogues of these molecules, including for example, but not limited to the N-protected derivatives of PG. Suitable protecting groups for PG include standard hydroxyl protecting groups known in the art, e.g., methoxymethyl (MOM), β-methoxyethoxymethyl (MEM), trialkylsilyl, triphenylmethyl (trityl), tert-butoxycarbonyl (t-BOC), ethoxyethyl (EE), f-MOC (methoxycarbonyl), TROC, etc. The protecting group(s) may be removed by using standard procedures generally known to those skilled in the art to give the desired PG derivatives (T. W. Green, Protective Groups in Organic Synthesis, Chapter 2, pages 10-69 (1981)).

Gastrins, pentagastrins, or analogues thereof are commercially available. In addition, synthetic protocols are well known. Thus, for example, PG can be chemically synthesized using well-known peptide synthesis methodologies (see, e.g. Barany and Merrifield Solid-Phase Peptide Synthesis; pp. 3-284 in The Peptides: Analysis, Synthesis, Biology. Vol. 2: Special methods in peptide synthesis, part a.; Merrifield et al. (1963) J. Am. Chem. Soc., 85: 2149-2156; and Stewart et al. (1984) Solid Phase Peptide Synthesis, 2nd ed. Pierce Chem. Co., Rockford, Ill.). Additionally, PG can be chemically synthesized, for example, by conjugation of a Boc-Ala residue to the tetrapeptide Trp-Met-Asp-PheNH₂.

The compositions of the present invention comprise PG or an analog thereof in an effective amount to achieve a pharmacological effect on the parietal cells without undue adverse side effects. The standard approximate amount of PG present in the compositions is preferably in an amount of 1-100 mg, more preferably 2-60 mg, and most preferably 4-40 mg of PG (or an equivalent amount of a PG analogue).

The oral compositions according to the present invention may further comprise one or more gastric acid stimulants in combination with PG or a PG analogue in order to obtain better activation of the parietal cells and synchronization between the activation of parietal cells and the absorption of PPI in blood in a meal-independent manner. The term “gastric acid stimulant” refers to any agent that is capable of stimulating gastric acid secretion via direct or indirect effect on parietal cells. Preferred gastric acid stimulants to be used in combination with PG or a PG analogue are small dicarboxylic and tricarboxylic acids such as succinic acid, maleic acid, citric acid and fumaric acid, or the salt thereof. The dicarboxylic or tricarboxylic acids are present in a pharmacological effective amount to stimulate acid secretion.

In a preferred embodiment, the oral composition of the present invention comprises a combination of PG in an oral dose ranging between 10 mg to 100 mg, more preferably between 20 mg to 40 mg and succinic acid in an amount ranging from about 10 mg to about 500 mg.

The compositions of the present invention further comprise a PPI that acts as an irreversible inhibitor of the gastric H⁺/K⁺-ATPase proton pump. The PPI used in the present invention can be any substituted benzimidazole compound having H⁺, K⁺-ATPase inhibiting activity. For the purposes of this invention, the term “PPI” shall mean any substituted benzimidazole possessing pharmacological activity as an inhibitor of H⁺,K⁺-ATPase, including, but not limited to, omeprazole, lansoprazole, pantoprazole, rabeprazole, dontoprazole, perprazole (s-omeprazole magnesium), habeprazole, ransoprazole, pariprazole, and leminoprazole in neutral form or a salt form, a single enantiomer or isomer or other derivative or an alkaline salt of an enantiomer of the same.

Examples of gastric H⁺/K⁺-ATPase proton pump inhibitors that may be used in the present invention are disclosed for example in U.S. Pat. No. 6,093,738 that describes novel thiadiazole compounds that are effective as proton pumps inhibitors. European Patent Nos. 322133 and 404322 disclose quinazoline derivatives, European Patent No. 259174 describes quinoline derivatives, and WO 91/13337 and U.S. Pat. No. 5,750,531 disclose pyrimidine derivatives, as proton pump inhibitors. Suitable proton pump inhibitors are also disclosed for example in EP-A1-174726, EP-A1-166287, GB 2 163 747 and W090/06925, W091/19711, W091/19712, W094/27988 and W095/01977.

The PPI particles in the compositions according to the present invention may be either coated or non-coated. The preparation of enteric-coated particles comprising a PPI such as Omeprazole is disclosed for example in U.S. Pat. Nos. 4,786,505 and 4,853,230.

The compositions of the present invention comprise a PPI in an effective amount to achieve a pharmacological effect or therapeutic improvement without undue adverse side effects. A therapeutic improvement includes but is not limited to: raising of gastric pH, reduced gastrointestinal bleeding, or improvement or elimination of symptoms. According to a preferred embodiment, the typical daily dose of the PPI varies and will depend on various factors such as the individual requirements of the patients and the disease to be treated. In general, the daily dose of PPI will be in the range of 1-400 mg. A preferred standard approximate amount of a PPI present in the composition is typically about 20-80 mg of omeprazole, about 30 mg lansoprazole, about 40 mg pantoprazole, about 20 mg rabeprazole, and the pharmacologically equivalent doses of the following PPIs: habeprazole, pariprazole, dontoprazole, ransoprazole, perprazole (s-omeprazole magnesium), and leminoprazole.

In a preferred embodiment, the compositions of the present invention further comprise one or more agents that preserve the availability of PG in the acidic gastric fluids. More specifically, the preservation agent maintains the stability or the solubility of PG in the gastric fluids. This enables PG to act locally in the stomach to activate the parietal cells. Such agents are preferably pH regulating agents, such as, buffering agents, alkaline agents or antacids that when dissolved in the gastric juice are capable of elevating the pH of the gastric fluids to a pH in which the gastric-residing peptidases are inhibited and at least significant proportion of PG remains soluble in the gastric fluids.

pH regulating agents to be used in the present invention include for example: sodium or potassium bicarbonate, magnesium oxide, hydroxide or carbonate, magnesium lactate, magnesium glucomate, aluminum hydroxide, aluminium, calcium, sodium or potassium carbonate, phosphate or citrate, di-sodium carbonate, disodium hydrogen phosphate, a mixture of aluminum glycinate and a buffer, calcium hydroxide, calcium lactate, calcium carbonate, calcium bicarbonate, and other calcium salts. It is noted that while sodium bicarbonate dissolves easily in water, calcium carbonate is water-insoluble and is slowly soluble only in acidic environment. Therefore, calcium carbonate may be useful when sustained dissolution of the pH regulating agent in the stomach is desired.

Other, examples of pH regulating agents to be used in the present invention include one or more of the following combinations: alumina, calcium carbonate, and sodium bicarbonate; alumina and magnesia; alumina, magnesia, calcium carbonate, and simethicone; alumina, magnesia, and magnesium carbonate; alumina, magnesia, magnesium carbonate, and simethicone; alumina, magnesia, and simethicone; alumina, magnesium alginate, and magnesium carbonate; alumina and magnesium carbonate; alumina, magnesium carbonate, and simethicone; alumina, magnesium carbonate, and sodium bicarbonate; alumina and magnesium trisilicate; alumina, magnesium trisilicate, and sodium bicarbonate; alumina and simethicone; alumina and sodium bicarbonate; aluminum carbonate, basic; aluminum carbonate, basic, and simethicone; aluminum hydroxide; calcium carbonate; calcium carbonate and magnesia; calcium carbonate, magnesia, and simethicone; calcium carbonate and simethicone; calcium and magnesium carbonates; magaldrate; magaldrate and simethicone; magnesium carbonate and sodium bicarbonate; magnesium hydroxide; magnesium oxide.

Preferably, the compositions of the present invention comprise one or more pH regulating agents in an effective amount to achieve a pharmacological effect. Specifically, the pH regulating agents in the composition are present in an amount sufficient to elevate the pH of the gastric fluids to a pH above the pH optima for proteases found in the stomach for a time period sufficient for PG to activate the parietal cells in the stomach. In a preferred embodiment, the pH regulating agents are present in an amount sufficient to elevate the pH of the gastric fluids to a pH above 5 for a time period ranging from 5 to 60 minutes, preferably for a time period ranging from 5 to 30 minutes. The quantity of pH regulating agents required in the compositions of the present invention will necessarily vary with several factors including the type of pH regulating agent used and the equivalents of base provided by a given pH regulating agent. In practice, the amount required to provide good availability of PG in the stomach is an amount which, when added to a solution of 200 milliliters of artificial gastric fluid (prepared according to the United States Pharmacopea (USP) guideline), raises the pH of that HCl solution to at least pH 5.0. Preferably, at least 100 milligrams, and more preferably at least 300, and most preferably at least 500 milligrams of the pH regulating agents are used in the pharmaceutical compositions of the invention.

The present invention further relates to a kit for chronic treatment of gastric-acid secretion-related disorders based on PPI, PG and/or a PG analogue and adjustable amounts of pH regulating agents. The kit comprises two different doses: an initial dose for the early stage of treatment containing an effective amount of PPI granules combined with high amount of buffering agents and a continuance dose containing comparable amount of PPI granules combined with low amount of buffering agents. The kit of the present invention provides the advantage of immediate-release PPI-based composition having fast absorption in blood and fast onset of anti-secretory effect with adjustable amount of buffer in order to minimize the risk of alkalosis.

In one embodiment, the kit of the present invention is formulated as a separate dosage form for the initial stage and the continuance stage of treatment. The kit comprises two different separate doses: an initial dose for the early stage of treatment containing an effective amount of PPI granules, PG and/or a PG analogue combined with high amount of pH regulating agents and a continuance dose containing comparable amount of PPI granules and PG and/or a PG analogue combined with lower amount of pH regulating agents. Specifically, the initial dose containing one or more pH regulating agents in an amount sufficient to raise the acidic pH of the stomach to a pH in which the stability of PG in the stomach is maintained, preferable to a pH above about 5.5-6.0. In a non-limiting example, sodium bicarbonate is used as the buffering agent and the its amount ranges from about 1000 mg to about 2000 mg, more preferable from about 1300 mg to about 1800 mg. The continuance dose contains one or more pH regulating agents in an amount sufficient to raise the gastric pH from about 4-5 to above about 5.5-6.0. When sodium bicarbonate is used as the pH regulating agent, its amount ranges from about 50 mg to about 500 mg, more preferable from about 50 mg to about 300 mg.

The buffering agent in the acute dose is preferably formulated as suspension tablet, effervescent tablet, chewable tablet or powder for suspension in order to provide fast relief from heartburns in patients. However, tablets or capsules are also possible as a dosage form for the buffering agents.

In another embodiment, the compositions of the present invention further comprise other agents that preserve the availability of PG in the acidic gastric fluids. For example, the compositions may comprise pepsin inhibitors such as the activated pentapeptide pepstatin and its derivatives, either of natural or synthetic origin. These inhibitors might decrease the degradation of PG by pepsin. Furthermore, the compositions may comprise mucolytic agents that reduce the viscosity of the gastric mucosa, thereby accelerating the ability of PG to reach the parietal cells. Such mucolytic agents are for example reducing agents such as N-acetyl cysteine, dithiothreitol, citric acid or mannitol. The compositions alternatively may also comprise a polymeric coating for PG, such as, an enteric-coating polymers to protect the PG from the acidic environment of the stomach.

The active ingredients of the present invention are preferably formulated in a single oral dosage form containing all active ingredients. The compositions of the present invention may be formulated in either solid or liquid form. It is noted that solid formulations are preferred in view of the improved stability of solid formulations as compared to liquid formulations.

In one embodiment, the PPI particles, PG and the one or more agents that preserve the availability of PG in the gastric fluids are formulated in a single solid dosage form such as multi-layered tablets, suspension tablets, effervescent tablets, powder, pellets, granules or capsules comprising multiple beads. In another embodiment, the active agents may be formulated in a single liquid dosage form such as suspension containing all active ingredients or dry suspension to be reconstituted prior to use.

In the single dosage form, the PPI particles and the PG particles may be coated with either enteric pH-dependent release polymer or non-enteric, time-dependent release polymer in order to synchronize between the local biological activity of PG in the stomach and the systemic effect of the PPI on parietal cells. For example, if coated PPI particles are used resulting in delayed absorption in blood, it is desirable that the PG particles be coated as well to delay its release. In one specific embodiment, the PPI particles are coated with a thick non-enteric layer so as the release of the PPI is preferably delayed by between, 20-80 min, more preferably 25-75 min, most preferably 30-60 min, and the PG particles are coated with a thin non-enteric polymer layer so as the release of PG is preferably delayed by 5-60 min, more preferably between 8-45 min, and most preferably 10-30 min. These conditions permit pre-activation of the parietal cells by PG prior to the achievement of a pharmacological PPI plasma concentration.

Non-limiting examples of suitable pH-dependent enteric polymers to be used in the present invention are: cellulose acetate phthalate, hydroxypropylnethylcellulose phthalate, polyvinylacetate phthalate, methacrylic acid copolymer, shellac, hydroxypropylmethylcellulose succinate, cellulose acetate trimellitate, and mixtures of any of the foregoing. A suitable commercially available enteric material, for example, is sold under the trademark Eudragit L 100-55. This coating can be spray coated onto the substrate.

Non-enteric time-dependent release polymers include, for example, one or more polymers that swell in the stomach via the absorption of water from the gastric fluid, thereby increasing the size of the particles to create thick coating layer. The time-dependent release coating generally possesses erosion and/or diffusion properties that are independent of the pH of the external aqueous medium. Thus, the active ingredient is slowly released from the particles by diffusion or following slow erosion of the particles in the stomach.

The erosion properties of the polymer in the stomach resulting from the interaction of fluid with the surface of the dosage form are determined mainly by the polymer molecular weight and the drug/polymer ratio. In order to ensure a delay of between about 10 min to about 60 min in the release of PG and PPI, it is recommended that the molecular weight of the polymer be in the range from about 10⁵ to about 10⁷ gram/mol. Furthermore, it is recommended that the PG or PPI/polymer ratio be in the range of about 2:3 to about 9:1, preferably about 3:2 to 9:1, and most preferably about 4:1 to 9:1.

Suitable non-enteric time-dependent release coatings are for example: film-forming compounds such as cellulosic derivatives, such as methylcellulose, hydroxypropyl methylcellulose (HPMC), hydroxyethylcellulose, and/or acrylic polymers including the non-enteric forms of the Eudragit brand polymers. Other film-forming materials may be used alone or in combination with each other or with the ones listed above. These other film forming materials generally include poly(vinylpyrrolidone), Zein, poly(ethylene glycol), poly(ethylene oxide), poly(vinyl alcohol), poly(vinyl acetate), and ethyl cellulose, as well as other pharmaceutically acceptable hydrophilic and hydrophobic film-forming materials. These film-forming materials may be applied to the substrate cores using water as the vehicle or, alternatively, a solvent system. Hydro-alcoholic systems may also be employed to serve as a vehicle for film formation.

Other materials which are suitable for making the time-dependent release coating of the invention include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose; and other materials known to those of ordinary skill in the art. Other lamina forming materials that can be used for this purpose include poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer.

Another approach for delaying the release of PG in the stomach is the use of floating particles having density lower than the gastric fluid, thereby delaying the release of PG from the particles. In one preferred embodiment, floating particles are obtained by the release of carbon dioxide within ethylcellulose-coated sodium bicarbonate beads upon contacting with the gastric juice. The release of carbon dioxide from the ethylcellulose-coated sodium bicarbonate core permits the buoyancy of the particles, thereby delaying the release of PG from the particles.

Other delayed gastric emptying approaches may be used in order to delay the release of PG in the stomach. These include the use of indigestible polymers or fatty acid salts that change the motility pattern of the stomach to a fed state, thereby decreasing the gastric emptying rate and permitting considerable prolongation of drug release (disclosed for example in Singh and Kim, J. of Controlled Release 63 (2000) 235-259).

In certain conditions, it is desirable to prolong the retention time of PG in the stomach by using dosage forms that unfold rapidly within the stomach to a size that resists gastric emptying. Such systems retain their integrity for an extended period and will not empty from the stomach at all until breakdown into small pieces occurs. Caldwell (Caldwell, L. J., Gardener, C. R., Cargill, R. C. (1988), U.S. Pat. No. 4,767,627) describes a cross shaped device made of erodible polymer and loaded with drug which is folded and inserted into a hard gelatin capsule. Following oral administration the gelatin shell disintegrates and the folded device opens out. With a minimum size of 1.6 cm and a maximum size of 5 cm it will not pass from the stomach through the pylorus until the polymer erodes to the point where the system is sufficiently small that it can be passed from the stomach.

An alternative approach to prolong the retention time of PG in the stomach is to use a hydrophilic erodible polymer system such as Poly(ethylene oxide) (Polyox) and Hydroxypropyl-methylcellulose (HPMC) that is of a convenient size for administration to humans. On imbibing fluid the system swells over a short period of time to a size that will encourage prolonged gastric retention, allowing sustained delivery of contained drug to absorption sites in the upper gastrointestinal tract. Because these systems are made of an erodible and hydrophilic polymer or polymer mixture, they readily erode over a reasonable time period to pass from the stomach. The time period of expansion is such that this will not occur in the esophagus and if the system passes into the intestine in a partially swollen state, the erodibility and elastic nature of the hydrated polymer will eliminate the chance of intestinal obstruction by the device.

In one specific example, the composition of the present invention is formulated as a single dosage form comprising multiple beads contained in hard or soft gelatin capsules. The capsules contain mixed population of beads selected from: beads comprising enteric-coated PPI or beads comprising PPI coated with time-dependent release polymer, beads comprising calcium carbonate and beads comprising ethylcellulose sodium bicarbonate beads coated with PG, calcium carbonate and hydroxypropyl methylcellulose. The cellulose-based polymer in the composition permits the floating of the PG beads, thus delaying the release of PG from the beads. The rate of PG release is determined by the thickness and the erosion rate of the hydroxypropyl methylcellulose.

In another specific example, the gelatin capsules contain mixed population of beads selected from: beads comprising enteric-coated PPI or beads comprising PPI coated with time-dependent release coating, beads comprising calcium carbonate and beads comprising alginate coated with PG, calcium carbonate and hydroxypropyl methylcellulose.

In yet another specific example, the gelatin capsules contain mixed population of beads selected from: beads comprising enteric-coated PPI, beads comprising PPI coated with time-dependent release polymer, beads comprising calcium carbonate and particles in the form of mini-tabs comprising PG, calcium carbonate and hydroxypropyl methylcellulose.

In yet another example, the compositions of the present invention are formulated as press-coat or double-layered tablets comprising enteric-coated PPI in one layer and PG, calcium carbonate and hydroxypropyl methylcellulose in a second layer.

In yet another example, the compositions of the present invention may be formulated as two layer non-aqueous semi-solid fill into hard gelatin capsules in which the PPI is solubilized in a lipid base (non-aqueous, quick release) which is liquid above room temperature but forms a semi-solid on cooling and can therefore be filled into hard gelatin capsules. A lipid soluble pH regulating agent such as an amine or a fine suspension of sodium bicarbonate may be included as well.

In one preferred embodiment, the single dosage form compositions of the present invention comprise a non-coated PPI instead of the enteric-coated PPI particles or the time-dependent release particles. The absorption of non-coated PPI in the upper portion of the small intestine is faster than the absorption of the coated PPI. Therefore, the use of non-coated PPI in the compositions permits more precise synchronization between the biological activity of PG in the stomach and the time period in which the PPI is active without the need for delaying the release of PG. Thus, according to various preferred embodiments, the compositions according to the present invention are formulated as double-layered tablets, press-coat tablets, effervescent tablets or suspension tablets comprising PG, non-coated PPI and one or more pH regulating agents.

In another preferred embodiment, the PPI granules are formulated as enteric-coated PPI granules. The enteric-coated PPI granules will provide an immediate release profile and fast absorption of PPI in blood due to the basic environment of the stomach. The basic environment rapidly induces the dissolving of the enteric-coating from the PPI granules, thereby permitting immediate release of the PPI from the granules and fast absorption in blood.

The active ingredients of the present invention may be formulated in a multiple oral dosage forms in which PG and the one or more agents that preserve the availability of PG in the gastric fluids are administered in a separate dosage form but in conjugation with the PPI. For example, PG and the one or more agents that preserve the availability of PG in the gastric fluids may be formulated in oral suspension or a solid dosage form such as capsules, tablets, suspension tablets, or effervescent tablets and the PPI may be formulated in a separate solid dosage form, preferably enteric-coated beads or time-dependent release beads contained in capsules or tablets.

When using multiple oral dosage forms, the PG and the one or more agents that preserve the availability of PG in the gastric fluids can be administered before, simultaneously with, or after the PPI. In sequential administration, there may be some substantial delay (e.g., minutes or even few hours) between the administration of PG and the PPI as long as the PG has exerted some physiological effect when the PPI is administered or becomes active. In a preferred embodiment, the PPI administered is in the enteric-coated or the time-dependent release form. According to this embodiment, it is preferable that the PPI administration precedes the PG administration in order to ensure that the PPI absorbed in the proximal part of the small intestine will be available for inhibiting the H⁺/K⁺-ATPase pumps while PG is still active in the stomach.

The active ingredients of the present invention may be incorporated within inert pharmaceutically acceptable beads. In this case, the drug(s) may be mixed with further ingredients prior to being coated onto the beads. Ingredients include, but are not limited to, binders, surfactants, fillers, disintegrating agents, alkaline additives or other pharmaceutically acceptable ingredients, alone or in mixtures. Binders include, for example, celluloses such as hydroxypropyl methylcellulose, hydroxypropyl cellulose and carboxymethyl-cellulose sodium, polyvinyl pyrrolidone, sugars, starches and other pharmaceutically acceptable substances with cohesive properties. Suitable surfactants include pharmaceutically acceptable non-ionic or ionic surfactants. An example of a suitable surfactant is sodium lauryl sulfate.

The particles may be formed into a packed mass for ingestion by conventional techniques. For instance, the particles may be encapsulated as a “hard-filled capsule” using known encapsulating procedures and materials. The encapsulating material should be highly soluble in gastric fluid so that the particles are rapidly dispersed in the stomach after the capsule is ingested.

In another embodiment, the active ingredients of the present invention are packaged in compressed tablets. The term “compressed tablet” generally refers to a plain, uncoated tablet for oral ingestion, prepared by a single compression or by pre-compaction tapping followed by a final compression. Such solid forms can be manufactured as is well known in the art. Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmaceutically compatible carriers. The manufacturing processes may employ one, or a combination of, four established methods: (1) dry mixing; (2) direct compression; (3) milling; and (4) non-aqueous granulation. Lachman et al., The Theory and Practice of Industrial Pharmacy (1986). Such tablets may also comprise film coatings, which preferably dissolve upon oral ingestion or upon contact with diluent.

Non-limiting examples of pH regulating agents which could be utilized in such tablets include sodium bicarbonate, alkali earth metal salts such as calcium carbonate, calcium hydroxide, calcium lactate, calcium glycerophosphate, calcium acetate, magnesium carbonate, magnesium hydroxide, magnesium silicate, magnesium aluminate, aluminum hydroxide or aluminum magnesium hydroxide. A particular alkali earth metal salt useful for making an antacid tablet is calcium carbonate.

In another alternative, the compositions of the present invention are formulated in compressed forms, such as suspension tablets and effervescent tablets, such that upon reaction with water or other diluents, the aqueous form of the composition is produced for oral administration. These forms are particularly useful for medicating children and the elderly and others in a way that is much more acceptable than swallowing or chewing a tablet. The present pharmaceutical tablets or other solid dosage forms disintegrate pH regulating agent with minimal shaking or agitation.

The term “suspension tablets” as used herein refers to compressed tablets which rapidly disintegrate after they are placed in water, and are readily dispersible to form a suspension containing a precise dosage of the PPI, the PG and the pH regulating agent. In one non-limiting example, the suspension tablets may comprise 20-40 mg omeprazole, 4 mg PG and about 1-4 grams of sodium or calcium bicarbonate as an pH regulating agent. To achieve rapid disintegration of the tablet, a disintegrant such as Croscarmellose sodium may be added to the formulation. The disintegrant may be blended in compressed tablet formulations either alone or in combination with microcrystalline cellulose, which is well known for its ability to improve compressibility of difficult to compress tablet materials. Microcrystalline cellulose, alone or co-processed with other ingredients, is also a common additive for compressed tablets and is well known for its ability to improve compressibility of difficult to compress tablet materials. It is commercially available under the Avicel trademark.

The suspension tablet composition may, in addition to the ingredients described above, contain other ingredients often used in pharmaceutical tablets, including flavoring agents, sweetening agents, flow aids, lubricants or other common tablet adjuvants, as will be apparent to those skilled in the art. Other disintegrants, such as crospividone and sodium starch glycolate may be employed, although croscarmellose sodium is preferred.

In addition to the above ingredients, the oral dosage forms described above may also contain suitable quantities of other materials, e.g. diluents, lubricants, binders, granulating aids, colorants, flavorants and glidants that are conventional in the pharmaceutical art. The quantities of these additional materials will be sufficient to provide the desired effect to the desired formulation. Specific examples of pharmaceutically acceptable carriers and excipients that may be used to formulate oral dosage forms are described in the Handbook of Pharmaceutical Excipients, American Pharmaceutical Association (1986), incorporated by reference herein.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### NaHCO₃ Preserves PG Stability in Artificial Gastric Fluid

The stability of PG in acidic pH in the presence of NaHCO₃ was tested in vitro using artificial gastric fluid. Artificial gastric fluid was prepared in accordance with U.S. Pharmacopoeia (USP) 2000 Ed., P. 235. For preparing 200 ml of gastric fluid, 0.4 g of NaCl and 0.64 g of Pepsin were dissolved in 16 ml 1M HCl and 184 ml of water. The pH of the gastric fluid was 1.2. Ten or twenty ml of 8.4% (1M) NaHCO₃ (final concentration 3.72 mg/ml or 7.12 mg/ml, respectively) and 16 ml of 250 ppm PG solution (0.25 mg/ml) were added to the solution. The concentration of PG in the final solution was 16 ppm. When indicated, Omeprazole granules were added as well (solutions B and C). In order to determine the stability of PG in the final solution over time, HPLC analysis was performed on samples taken at the following time points post preparation: 0′ (immediately following preparation), 5′, 10′, 20′, 40′, 60′. To stop the reaction, the pH was adjusted to 7.5-8.5 using NH₄OH.

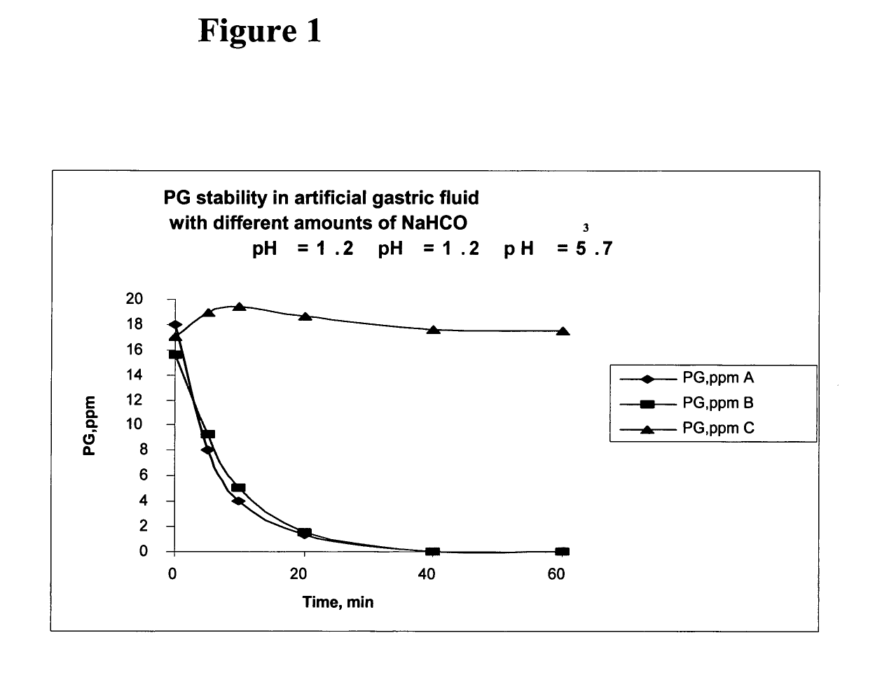

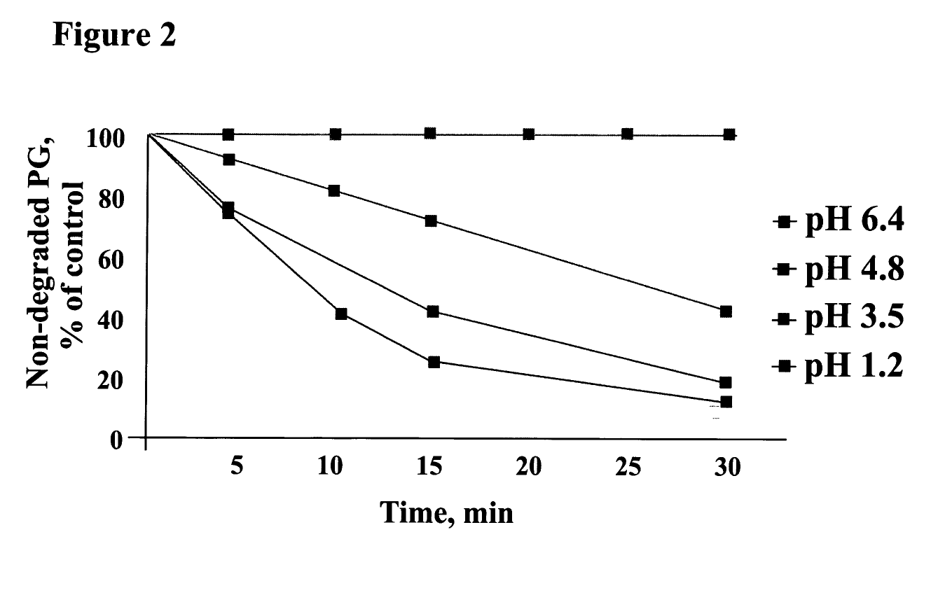

As demonstrated in FIG. 1, fast degradation of PG was observed in solutions A and B that comprise PG in the presence of 3.72 mg/ml of NaHCO₃ (pH 1.2). However, PG remained stable for 1 h in solution C that comprises 7.12 mg/ml of NaHCO₃ (pH 5.7). These results indicate that the addition of an pH regulating agent such as NaHCO₃ in a concentration sufficient to elevate the pH above 5.0 prevents the degradation of PG by pepsin. FIG. 2 further demonstrates that at least 80% of PG remains non-degraded for at least 15 min in pH 4.8.

A. Formulation Description—Tablets Containing Non-Enteric-Coated Omeprazole:

### Example 2

### Press-Coated or Double-Layered Tablets Comprising PG, Non-Enteric-Coated Omeprazole, Sodium Bicarbonate and Calcium Carbonate

Press-coated or double-layered tablets are formulated as a single dosage form in which each tablet containing the following ingredients:

| | |
|---|---|
| Omeprazole (powder) | 40 mg |
| PG | 4 mg |
| NaHCO₃ | 500 mg |
| CaCO₃ | 500 mg |
| Croscarmellose sodium | |
| hydroxypropyl methylcellulose (HPMC) | |
| Microcrystalline cellulose (Avicel) | |
| Magnesium stearate | |
| Starch | |

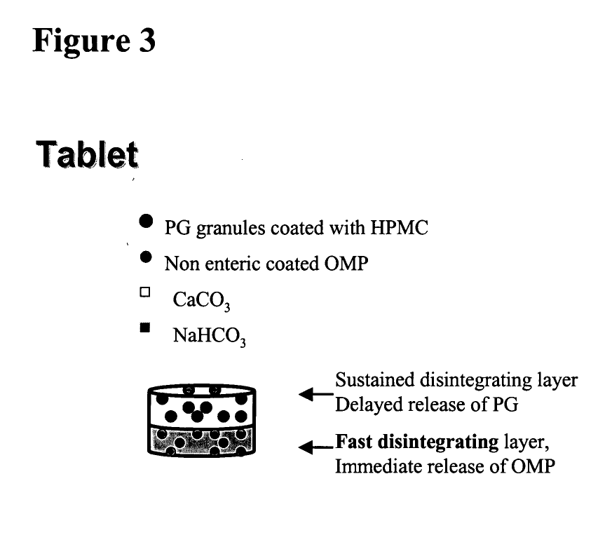

Press-coated or double-layered tablets are prepared in a two-step process. For a single tablet, 4 mg PG, 250 mg calcium carbonate and microcrystalline cellulose are mixed and pre-compressed into the first layer of the tablet. The layer containing the PG is further coated with a thin layer of HPMC that permits a delay of 10-15 min in the release of PG from the tablet. For the second layer, 40 mg of non-enteric-coated omeprazole powder together with 500 mg NaHCO3, 250 mg CaCO₃ and the appropriate binders are compressed onto the PG layer to form the second layer of the tablet. The second layer of the tablet disintegrates immediately after digestion to permit prompt release of omeprazole. A schematic illustration of a double-layered tablet comprising PG, non-enteric-coated omeprazole, sodium bicarbonate and calcium carbonate is presented in FIG. 3.

### Example 3

### Fast Disintegrating Tablets Comprising PG, Non-Enteric-Coated Omeprazole, Sodium Bicarbonate and Calcium Carbonate

Fast disintegrating tablets are formulated as a single dosage containing the following ingredients:

| | |
|---|---|
| Omeprazole (powder) | 40 mg |
| PG | 4 mg |
| NaHCO₃ | 500 mg |
| CaCO₃ | 500 mg |
| Croscarmellose sodium | |
| Microcrystalline cellulose | |
| Magnesium stearate | |
| Starch | |

Non-enteric-coated omeprazole (40 mg), PG (4 mg), NaHCO3, CaCO₃, Croscarmellose sodium, Microcrystalline cellulose and Magnesium stearate are mixed and the resulting mixture is compressed into tablets using standard tablet pressing to yield a fast disintegrating tablet (intravescent).

### Example 4

### Effervescent Sacs Comprising PG, Enteric-Coated Omeprazole, and Sodium Bicarbonate

Effervescent tablets are formulated as a single dosage containing the following ingredients:

| | |
|---|---|
| Omeprazole | 40 mg |
| PG | 4 mg |
| NaHCO₃ | 958 mg |
| Citric acid | 832 mg |
| Potassium carbonate | 312 mg |
| Magnesium stearate | |
| Starch | |

Enteric-coated omeprazole (40 mg) and PG (4 mg) are placed into a mortar and triturated with a pestle to a fine powder. Sodium bicarbonate, citric acid, potassium carbonate and all other excipients are added to the mixture to form a homogeneous mixture of effervescent powder. The resulting powder is mixed with 40 mg enteric-coated omeprazole and packed in packets of unit dose.

B. Formulation Description—Multi Particulate Capsules Containing Coated Omeprazole:

### Example 5

### Capsules Comprising Ethylcellulose-PG Beads, Enteric-Coated Omeprazole Beads, and Calcium Carbonate

This example illustrates the steps involved in manufacturing multi particulate hard gelatin capsules. Hard gelatin capsules are formulated as a single dosage form comprising mixed population of particles. Each capsule contains the following ingredients:
40 mg omeprazole as enteric-coated beads
4 mg PG loaded on ethylcellulose-coated sodium bicarbonate beads
600 mg calcium carbonate (CaCO₃)
hydroxypropyl methylcellulose (HPMC)

PG solution is prepared by dissolving PG in ammonium carbonate buffer pH 8. The PG solution is sprayed on the ethylcellulose-coated sodium bicarbonate beads in a fluidized bed apparatus. After drying, the PG-sodium bicarbonate beads are further coated with CaCO₃ and with hydroxypropyl methylcellulose (HPMC) to form the final PG particles. The final PG particles are packed together with enteric-coated omeprazole beads and calcium carbonate powder into size 0 hard gelatin capsules in an amount corresponding to 40 mg omeprazole, 4 mg PG and 600 mg calcium carbonate per capsule.

Upon dissociation of the gelatin capsules in the gastric juice of the stomach, the HPMC layer of the PG-containing beads expands and the gastric acid reacts with sodium bicarbonate to form CO₂ inside the bead core. The release of carbon dioxide from the ethylcellulose-coated sodium bicarbonate core permits the buoyancy of the particles, thereby delaying the release of PG and calcium carbonate from the particles. The rate of PG release is determined by the thickness and the erosion rate of the HPMC layer of the PG beads. CaCO₃ increases the gastric pH for a prolonged period of time, to protect PG upon release. The enteric-coated omeprazole beads pass the stomach and omeprazole is absorbed in the upper part of the small intestine without any delay.

### Example 6

### Capsules Comprising Alginate-PG Beads, Enteric-Coated Omeprazole Beads, and Calcium Carbonate

Hard gelatin capsules are formulated as a single dosage form comprising mixed population of particles. Each capsule contains the following ingredients:
40 mg omeprazole as enteric-coated beads
4 mg PG loaded on alginate particles
600 mg calcium carbonate (CaCO₃)
hydroxypropyl methylcellulose (HPMC)

Alginate particles are made by dropping an alginate solution into calcium chloride solution following by freeze-drying to yield alginate particles. The PG solution prepared as in Example 5 is sprayed on the alginate particles in a fluidized bed apparatus. After drying, the PG-alginate beads are further coated with CaCO₃ and with hydroxypropyl methylcellulose (HPMC) to form the final PG particles. The final PG particles together with the enteric-coated omeprazole beads and calcium carbonate powder are packed into size 0 hard gelatin capsules in an amount corresponding to 40 mg omeprazole, 4 mg PG and 600 mg calcium carbonate per capsule.

Upon dissociation of the gelatin capsules in the stomach, the PG beads are expanded due to the contact of the HPMC layer with the gastric juice. The freeze-dried alginate particles permit the buoyancy of the particles due to their low density thereby delaying the release of PG from the particles. The rate of PG release is determined by the thickness and the erosion rate of the HPMC layer of the PG beads. The enteric-coated omeprazole beads pass the stomach and omeprazole is absorbed in the upper part of the small intestine without any delay.

### Example 7

### Capsules Comprising Sucrose-PG Beads, Enteric-Coated Omeprazole Beads, and Calcium Carbonate

Hard gelatin capsules are formulated as a single dosage form comprising mixed population of particles. Each capsule contain the following ingredients:
40 mg omeprazole as enteric-coated beads
4 mg PG loaded on inert sugar beads
600 mg calcium carbonate (CaCO₃)
hydroxypropyl methylcellulose (HPMC)

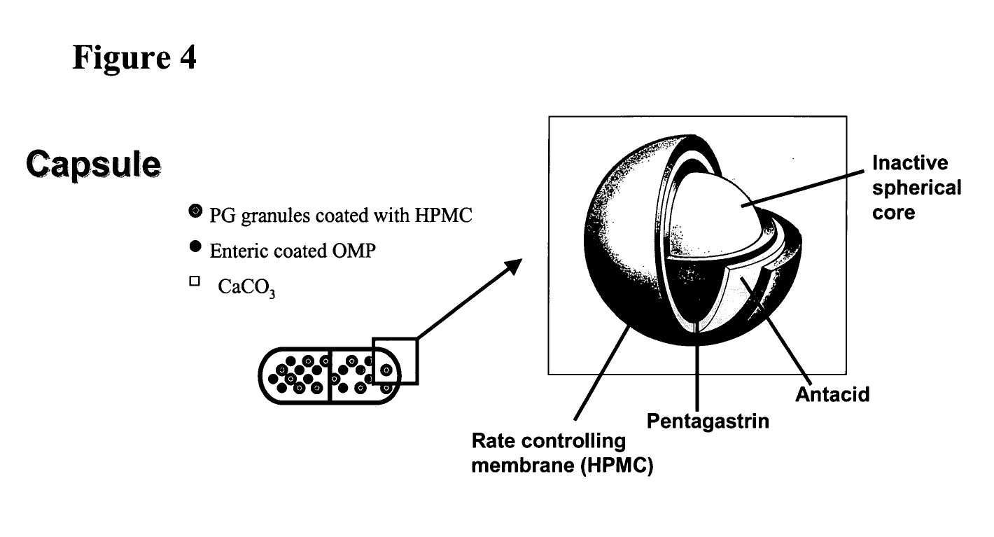

The PG solution is sprayed on inert sugar pellets (Nu-Pareils, 25/30) in a fluidized bed apparatus. After drying, the PG-sugar beads are further coated with CaCO₃ and with hydroxypropyl methylcellulose (HPMC) to form the final PG particles. A schematic illustration of the PG granules is presented in FIG. 4. The final PG particles together with the enteric-coated omeprazole beads and calcium carbonate powder are packed into size 0 hard gelatin capsules in an amount corresponding to 40 mg omeprazole, 4 mg PG and 600 mg calcium carbonate per capsule.

Upon dissociation of the gelatin capsules in the stomach, the PG beads are expanded due to the contact of the HPMC layer of the PG-containing beads with the gastric juice, thereby delaying the release of PG from the particles. The rate of PG release is determined by the thickness and the erosion rate of the HPMC layer of the PG beads. The enteric-coated omeprazole beads pass the stomach and omeprazole is absorbed in the upper part of the small intestine without any delay.

### Example 8

### Hard Gelatin Capsules Comprising HPMC-PG Minitabs, Enteric-Coated Omeprazole Beads, and Calcium Carbonate

Hard gelatin capsules are formulated as a single dosage form comprising mixed population of particles. Each capsule contains the following ingredients:
40 mg omeprazole as enteric-coated omeprazole beads
4 mg PG loaded on inert sugar beads
600 mg calcium carbonate (CaCO₃)
hydroxypropyl methylcellulose (HPMC)

PG is granulated in combination with HPMC and CaCO₃ and compressed into mini-tabs. The mini-tabs possess the ability of fast swelling upon contact with the gastric juice of the stomach, thereby enabling gastric retention. The release of PG into the stomach is controlled by the erosion rate of the polymeric matrix of the swelled mini-tabs. The PG mini-tabs together with the enteric-coated omeprazole beads are packed into size 0 hard gelatin capsules in an amount corresponding to 40 mg omeprazole, 4 mg PG and 600 mg calcium carbonate per capsule.

### Example 9

### Multi Particulate Capsules Containing Omeprazole and PG Beads Coated with Non-Enteric Time-Dependent Release Coating

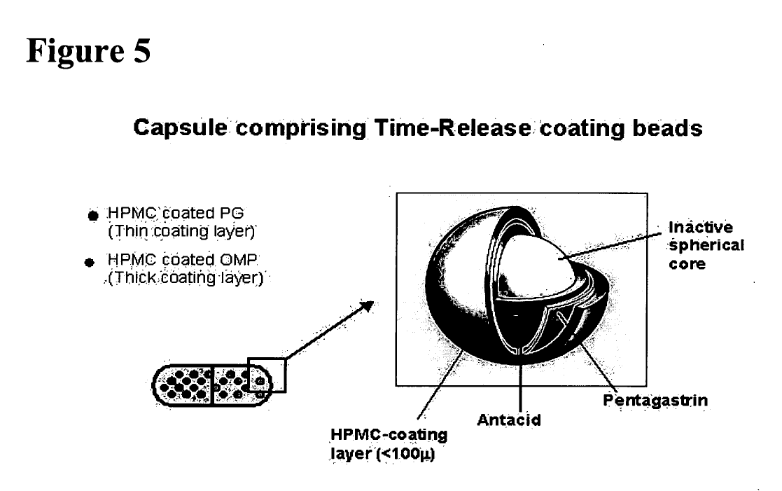

This example illustrates the steps involved in manufacturing multi particulate hard gelatin capsules. Capsules are formulated as a single dosage form comprising mixed population of particles: PG beads coated with time-dependent release coating, omeprazole beads coated with time-dependent release coating, and calcium carbonate. A schematic illustration of the capsule is present in FIG. 5. Each capsule contains the following ingredients:

40 mg omeprazole beads coated with thick HPMC layer

4 mg PG loaded on sugar spheres and coated with thin HPMC layer

600 mg calcium carbonate (CaCO₃)

The composition of the coating is designed such that the core is rapidly disintegrated into an aqueous environment when the media come into contact with the core. For this purpose Sugar sphere will be coated with an antacid (NaHCO₃ or CaCO₃) layer. PG solution is prepared by dissolving PG in ammonium carbonate buffer pH 8. The PG solution is sprayed on to the above antacid-coated beads in a fluidized bed apparatus. After drying, the beads are further coated with a thin layer of HPMC to create PG particles with approx. 10 min delayed release. Omeprazole is layered over the antacid-coated Sugar spheres and is covered with a thick time-release HPMC coating. A disintegrant also may be added to the core of the particle to facilitate the prompt release of omeprazole after the HPMC is dissolved. The coated Omeprazole beads are aimed to pass the stomach and are absorbed at the upper parts of the small intestine after the HPMC is dissolved and the Omeprazole is released at once. The final PG particles are packed together with the omeprazole beads and calcium carbonate powder into size 0 hard gelatin capsules in an amount corresponding to 40 mg omeprazole, 4 mg PG and 600 mg calcium carbonate per capsule. The rate of PG and OMP release is determined by the thickness and the erosion rate of the HPMC layer of the beads. CaCO₃ increases the gastric pH for a prolonged period of time, to preserve PG upon release.

C Formulation Description—Tablets Containing Enteric-Coated Omeprazole:

### Example 10

### Press-Coated Tablets Comprising PG, Enteric-Coated Omeprazole Beads, and Calcium Carbonate

Press-coated tablets are formulated as a single dosage form containing the following ingredients:
40 mg omeprazole as enteric-coated omeprazole beads
4 mg PG granules
Calcium carbonate
hydroxypropyl methylcellulose (HPMC)

Press-coated tablets are prepared in a two-step process. For a single tablet, 4 mg PG, 900 mg calcium carbonate and HPMC are mixed and pre-compressed into the central core of the tablet. 40 mg of enteric-coated omeprazole beads are press-coated onto the PG core to form the external layer of the tablet. The final tablet is composed of controlled-release PG core layer and immediate release outer layer of omeprazole enteric-coated beads. In another example, the active ingredients are compressed into double-layered tablet wherein the first layer comprises 4 mg PG, 900 mg calcium carbonate and HPMC and the second layer comprises 40 mg of enteric-coated omeprazole beads.

The compressed tablet may include one or more of the following excipients: lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmaceutically compatible carriers.

### Example 11

### Fast Disintegrating Tablets Comprising PG, Enteric-Coated Omeprazole Beads and Calcium Carbonate

Fast disintegrating suspension tablets are formulated as a single dosage containing the following ingredients:
40 mg omeprazole as enteric-coated omeprazole beads
4 mg PG granules
900 mg calcium carbonate
Croscarmellose sodium
Microcrystalline cellulose
Magnesium stearate
hydroxypropyl methylcellulose (HPMC).

PG granules are coated with CaCO₃ and with hydroxypropyl methylcellulose (HPMC) to form the final PG particles. The final PG particles are mixed with enteric-coated omeprazole beads and the excipients listed above and the resulting mixture is compressed into tablets using standard tablet pressing. The resulting tablets possess rapid disintegration time and may be swallowed with water for fast disintegration in the stomach.

Upon disintegration of the suspension tablet, the PG particles are expanded due to the contact of the HPMC layer of the PG-containing beads with aqueous environment, thereby delaying the release of PG from the particles. The rate of PG release is determined by the thickness and the erosion rate of the HPMC layer of the PG beads. The enteric-coated omeprazole beads pass the stomach and omeprazole is absorbed in the upper part of the small intestine without any delay.

D In vivo Experiments

Example 12

### Stimulation of Gastric Acid Secretion Following Oral Administration of PG in Rats

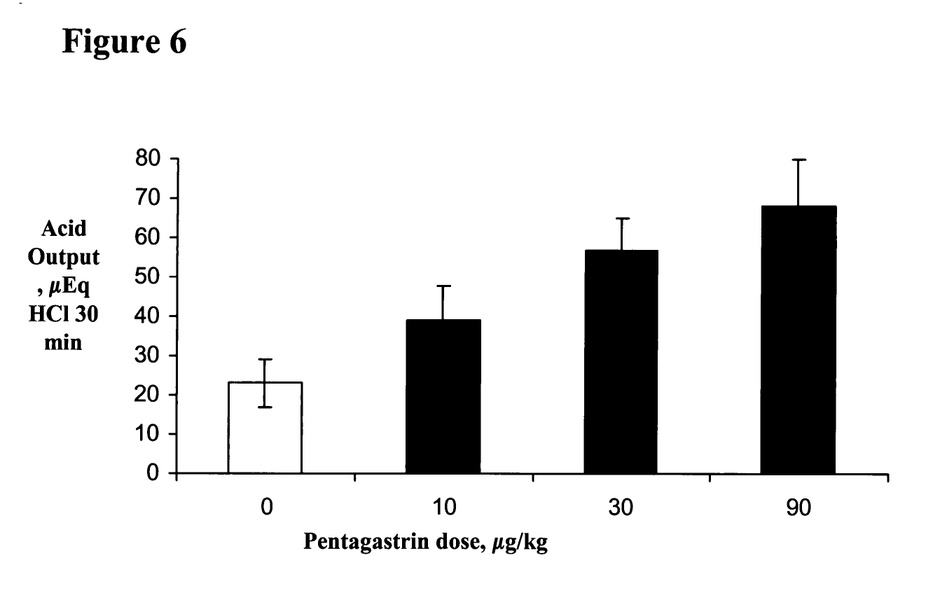

Inhibition of gastric acid secretion by a combination of PG and PPI is based on the ability of orally administered PG to trigger acid secretion locally within the stomach. To address this issue anesthetized rats were administered (per os) with increasing amounts of PG and gastric acid secretion was monitored in a pylorus-ligated stomachs. Increasing amounts (10, 30, and 90 μg/kg) of PG were administered by oral gavage to pylorus-ligated rats. Following 30 min treatment, gastric juice was collected from the gastric lumen, and acid concentration was determined by titration with NaOH and total acid output expressed in μEq HCl was calculated by multiplying the sample volume by the acid concentration. Results are expressed as means±SEM of 7-8 animals from each experimental group. As demonstrated in FIG. 6, orally administered PG significantly enhanced gastric acid secretion in a dose-dependent manner, suggesting that orally administered PG successfully induces gastric acid secretion in a local manner.

### Example 13

### The Effect of PG Administered with Omeprazole on Intragastric pH

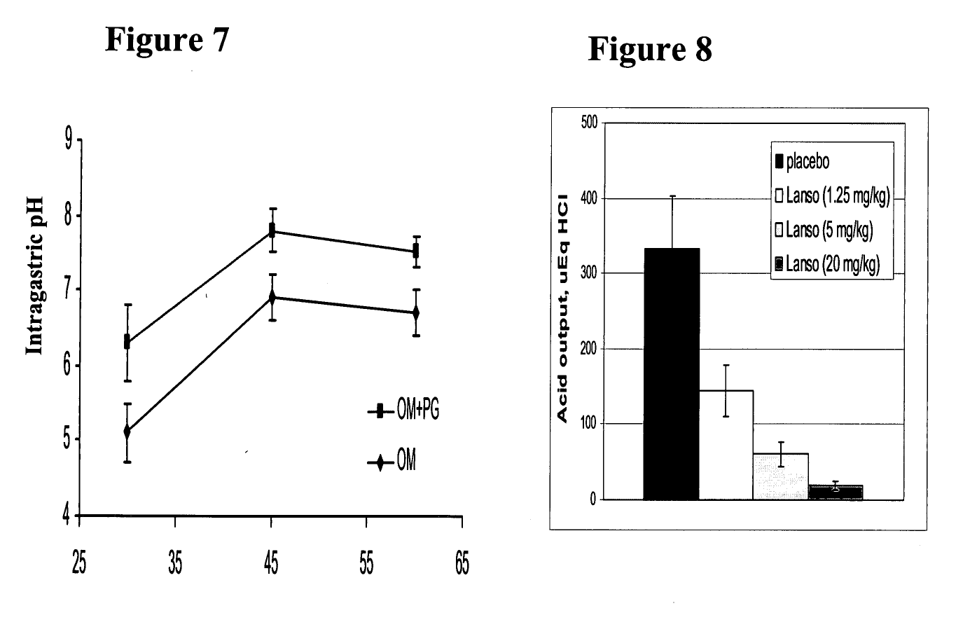

To test the effect of the PG-PPI combination on suppression of gastric acid secretion, anesthetized rats were subjected to intragastric injection of either omeprazole (10 mg/kg) alone or in combination with PG (350 μg/kg). Rats treated with the combination received PG 15 minutes before omeprazole. The gastric juice was collected by suction at 30, 45, and 60 minutes after the treatment and an effect of drugs on gastric acid secretion was detected by monitoring pH. The data demonstrated that the intragastric pH value at all time points was markedly higher in rats treated with combination of PG and omeprazole than with omeprazole alone (FIG. 7). These results indicate that PG enhances the anti-secretory activity of PPI in rats.

### Example 14

### Lansoprazole Inhibits Gastric Acid Secretion in Conscious Animals in a Dose-Dependent Manner

In this experiment, a different model of pylorus-ligated rats that permits the analysis of the effect of drugs on gastric acid secretion in conscious animals was used. This model eliminates the effect of anesthesia on gastric acid secretion. The study drugs alone or in combination were administered per os. One or two hours later the animals were anesthetized using anesthetic gas machine for a short period (5 minutes) that is sufficient to perform the pylorus ligation procedure and to close the abdomen. The animals were then placed back into its cage for recovery. Several hours later the animals were sacrificed, the ligature was placed around the esophagus, the stomach removed and gastric content was collected. Following centrifugation the gastric juice samples were automatically titrated with 0.01 N NaOH to endpoint pH 7 and titratable acid output was calculated.

Lansoprazole was administered by oral gavage as a simplified suspension (SLS). SLS was prepared as follows: the content of one 30 mg capsule (Zoton) was suspended in 8.4% sodium bicarbonate. Rats were treated with three doses of Lansoprazole (20, 5 and 1.25 mg/kg) 2 hours before pylorus ligation. 8.4% NaHCO3 was administered into the control group as a placebo. FIG. 8 demonstrates that Lansoprazole inhibited the gastric acid secretion in a dose-dependent manner.

### Example 15

### The Effect of Lansoprazole Administered in Combination with PG on Gastric Acid Secretion in Conscious Pylorus-Ligated Rats

In this experiment, rats were treated with SLS at a dose 5 mg/kg either 15 minutes before (A) or after (B) PG (300 μg/kg). The control rats were injected with combination of 8.4% NaHCO3 and PG-vehicle as a placebo. All drugs were administered by oral gavage 2 hours before pylorus ligation. The gastric juice was collected during 3 hours. Data is presented as mean±SEM. Number of animals is 8-9 in each experimental group.

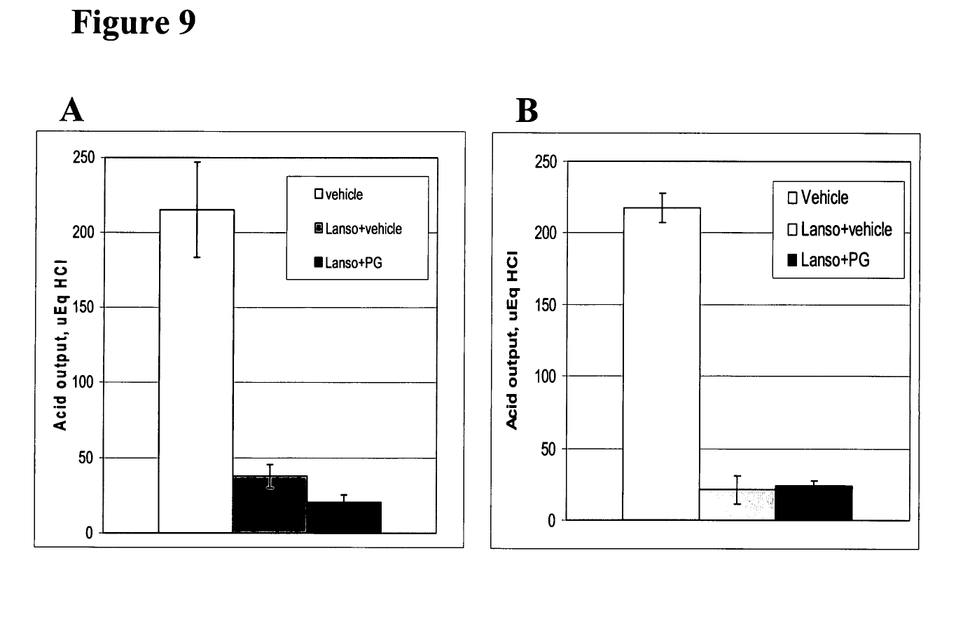

As can be seen in FIG. 9A the administration of SLS 15 minutes before PG led to a greater extent of acid inhibition as compared to Lansoprazole alone, whereas acid output in rats pretreated with PG and then treated with SLS did not differ from that of Lansoprazole alone-treated rats (FIG. 9B). These results indicate that PG increases the efficacy of Lansoprazole in the blockade of gastric acid secretion. Moreover, the timing between the two compounds is important in order to get increased effectiveness of PG/Lansoprazole combined treatment.

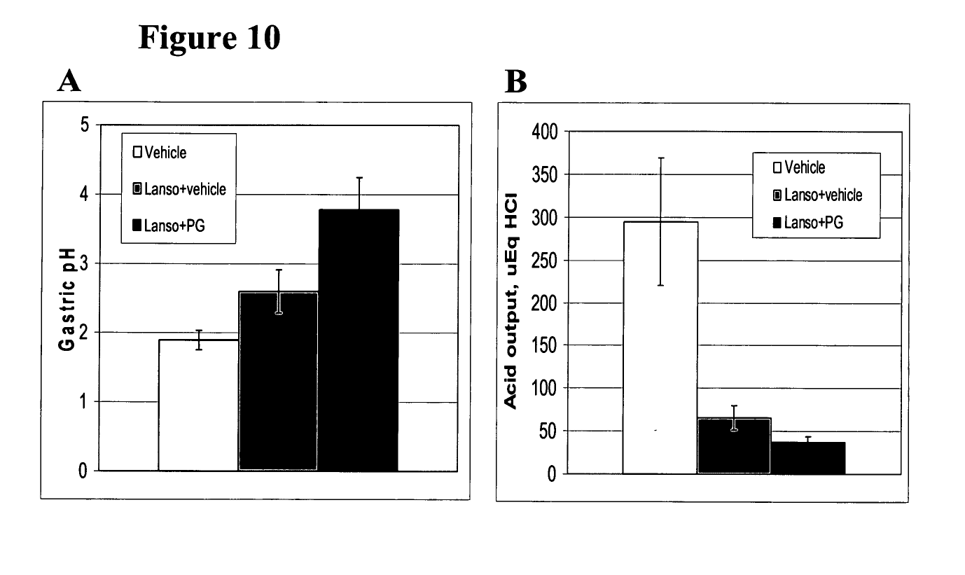

In another experiment, rats were treated once daily during 3 consecutive days with either SLS at a dose 2.5 mg/kg and vehicle or SLS and PG (300 μg/kg). SLS was administered 15 minutes before PG or vehicle. The control rats were injected with combination of 8.4% NaHCO3 and PG-vehicle as a placebo. All drugs were administered by oral gavage. The pylorus ligation was performed on third day 2 hours following treatment. The gastric juice was collected during 3 hours. Data is presented as mean±SEM. Number of animals is 8 in each experimental group. As demonstrated in FIGS. 10A and 10B, administration of SLS in combination with PG during 3 consecutive days resulted in significantly higher intragastric pH as compared to SLS alone. Similarly, the gastric acid secretion in rats treated with SLS/PG combination for three consecutive days was lower than that following administration of SLS alone.

### Example 16

### The Effect of a CCK-B Antagonist on PG-Mediated Gastric Acid Secretion in Rats

As PG is a gastrin hormone homologue, its local effect is thought to be mediated via gastrin pathway, i.e. an activation of gastrin receptor (CCKB). To test this hypothesis the effect of the specific CCKB antagonist (Itriglumide) on PG-mediated acid secretion in rats was examined.

In this study, rats were anesthetized with Ketamine and Domitor mixture and provided with 20 mg/kg of Itriglumide that was administered intraduodenally (i.d.). Following 15 min, gastric pylorus was ligated and 300 μg/kg PG was administered into the stomach (i.g.). After 30 min, gastric juice was obtained, centrifuged and the volume and pH of the supernatants were measured. The acid concentration (titratable acidity) was analyzed by titration the gastric juice samples with NaOH and total acid output expressed in μEq HCl was calculated by multiplying the sample volume by the acid concentration. As revealed from the results presented in Table 1 below, intraduodenal injection of CCKB antagonist (ant.) inhibits the local effect of PG on gastric acid secretion in rats.

TABLE 1

| | Acid Output | |
|---|---|---|
| Group | MEAN | ±SEM |
| PG (i.g.), 300 ug/kg | 60.056 | 10.43 |
| CCKB ant. (i.d.) 20 mg/kg | 15.24 | 2.82 |
| Placebo of PG (i.g.) - | 19.25 | 3.03 |
| NH4HCO3 | | |
| Placebo of CCKB ant. - | 12.93 | 1.55 |
| saline (i.d.) | | |
| PG (i.g.), 300 ug/kg and | 22.884 | 2.70 |
| CCKB ant. (i.d.) 20 mg/ml | | |
| PG (i.g.), 300 ug/kg and | 51.74 | 9.35 |
| Placebo of CCKB ant. - | | |
| saline (i.d.) | | |
| Student t-test | | |
| PG vs.ant.+PG | P = 0.0023 | |
| | P = 0.0042 | |
| | P = 0.0016 | |

### Example 17

### The Effect of Intraduodenal Injection of PG on Acid Secretion in Anesthetized Pylorus-Ligated Rats

The effect of intraduodenal injection of PG on acid secretion in anesthetized pylorus-ligated rats was examined. In this study, 300 μg/kg PG was administered intraduodenaly in anesthetized pylorus-ligated rats and the level of gastric acid secretion was determined 30 minutes later. Gastric juice was obtained, centrifuged and the volume and pH of the supernatants were measured. The acid concentration (titratable acidity) was analyzed by titration gastric juice samples with NaOH and total acid output expressed in μEq HCl was calculated by multiplying the sample volume by the acid concentration. As a control the equal amount of PG was injected intragastrically and the effect of PG on gastric secretion was determined. As demonstrated in Table 2, both intragastric and intraduodenal injection of PG induce gastric acid secretion in anesthetized pylorus-ligated rats.

TABLE 2

| | Acid Output | |
|---|---|---|
| Group | MEAN | ±SEM |
| PG (i.g.), 300 ug/kg | 45.89 | 6.37 |
| Placebo (i.g.) | 12.46 | 2.65 |
| PG (i.d.), 300 ug/kg | 42.26 | 6.95 |
| Placebo (i.d.) | 11.65 | 1.44 |
| Student t-test | | |
| G (i.g.) vs. Placebo | P = 0.000125 | |
| | P = 0.000243 | |
| | P = 1.981 × 10⁵ | |

E Clinical Studies

### Example 18

### A Satellite Study for Testing the Safety and Efficacy of a Single—Dose Co—Treatment with Oral Pentagastrin and Omeprazole in Healthy Subjects

Rationale & Objectives:

Proton pump inhibitors are the most effective class of drugs for inhibiting acid secretion. Their efficacy depends on the number of “activated” parietal cell H⁺,K⁺ ATPase pumps at the time in which PPIs enter the canalicular space due to acid-dependent accumulation. Subcutaneous PG was shown to be an effective agent for stimulating parietal cells and activating proton pumps. Oral PG was shown to stimulate gastric acid secretion and thereby to potentiate the effect of PPI in a rat model. However, no information regarding the effect of oral PG is available in human subjects. The objective of this study was to test the safety and efficacy of a single administration of two oral doses of pentagastrin (4 mg and 12 mg), sodium bicarbonate and a proton pump inhibitor (Omeprazole) in H. pylon negative volunteers.

METHODOLOGY: Number of subjects/patients: The study was designed in two phases. In the first phase, 9 eligible subjects were treated with 4 mg Pentagastrin in combination with Omeprazole (40 mg) and sodium bicarbonate in an open manner. This phase was aimed to test the safety and efficacy of the drug combination. Two subjects were not analyzed for efficacy due to technical difficulties with gastric pH measurements. After the safety analysis was completed and as safety criteria were met, an open crossover study (second phase) with 12 subjects treated with the higher dose of Pentagastrin (12 mg), Omeprazole and sodium bicarbonate was performed. In the cross-over treatment, the 12 subjects received Omeprazole and sodium bicarbonate only. The second phase was aimed to test the safety of higher dose of PG and to compare the efficacy of treatments in controlling gastric acid secretion. One subject did not participate in the placebo crossover study. Two subjects received the same 12 mg treatment twice due to technical difficulties with gastric pH measurements during the first treatment. Treatments were spaced at least 14 days apart.

Diagnosis and main criteria for inclusion: Healthy volunteers with normal lab tests and no previous gastrointestinal surgery or history of significant medical illness, who were H. pylori negative as confirmed by a negative ¹³C urea breath test.

Duration of treatment: A single treatment was administered and pH was monitored for 24 hours. The 12 subjects in the open crossover study had a period of at least 14 days between treatments.

Drug Doses and Mode of Administration:

Oral administration of PG solution—either one vial of 16 ml (for the 4 mg treatment) or three vials (for the 12 mg treatment). The 0 mg PG group

Omeprazole 40 mg capsules (Losec—Abic Ltd) as enteric-coated granules suspended in 100 ml apple juice. Oral administration.

Sodium bicarbonate 30 ml 8.4% solution (B. Braun)—Oral administration.

Criteria for Evaluation:

Primary Efficacy Endpoints:

Percent of post-dose daytime and nocturnal time of gastric pH above 4.

Secondary Efficacy Endpoints

Percent of post-dose daytime and nocturnal time of gastric pH above 3, 5, or 6.

Safety:

Incidence of adverse events related to study medication;

Changes in vital signs and physical examination;

Significant changes associated with the study medication in hepatic and renal function as determined by biochemistry and urinalysis;

Significant hematological changes associated with study medication;

Maintenance of normal ECG;

Percent of time in which gastric pH is below 1.5.

Statistical Methods:

Results were analyzed with repeated-measures (mixed model) ANOVA (analysis of variance) comparing the treatment (12 mg PG) vs. placebo (0 mg PG) arms. This specific method was chosen rather than the paired t—tests originally specified in order not to loose one subject who did not participate in the placebo crossover arm.

One method was used for evaluating the safety criteria. This was the length of time where gastric pH was below 1.5. Results were analyzed with repeated measure (mixed model) ANOVA (analysis of variance) comparing treatment vs placebo arms. The reason for using (mixed model) ANOVA was, as above, to include the one patient who did not participate in the placebo crossover arm.

Adverse events, physical examination, vital signs, blood chemistry, hematology and urinalysis were summarized. Results are presented descriptively.

ANOVA (mixed model) was used to evaluate the results in the post hoc analysis.

Efficacy Results:

A group of nine subjects received a single dose of 4 mg PG, 40 mg Omeprazole and sodium bicarbonate and their gastric pH was subsequently monitored. Two subjects were excluded from the efficacy analysis due to technical failure during data acquisition. The percent of time with gastric pH above 4 in this group was 35.7±22.4 (mean±SD). A further 12 subjects served in the placebo-controlled test with either 12 mg or 0 mg (placebo) PG in combination with 40 mg Omeprazole and sodium bicarbonate. In two subjects the tracing was repeated in order to assure accuracy of the recording. The primary efficacy endpoint of percent of post-dose daytime and nocturnal time of gastric pH above 4 was not significantly different between the placebo or 12 mg PG treatments. There was however a non-statistically significant dose-dependent increase in the percent of time with gastric pH above 4 with the 12 mg group (0 mg PG: 34.8±4.2 versus 12 mg PG: 41.3±3.9, mean±SEM). The secondary efficacy endpoints of percent of post-dose daytime and nocturnal time of gastric pH above 3, 5, 6 were not significantly different between the treatment groups although in most cases the values of the 12 mg PG were higher than the placebo.

A post-hoc analysis was performed based on the pharmacokinetic measurements of Omeprazole levels in plasma and technical evaluation of the pH monitor tracing. In this analysis, only subjects who had technically acceptable pH tracings and omperazole levels that were higher than about 60 ng/ml during 180 minutes after Omeprazole intake were included. Using these criteria, eight subjects in the group of volunteers who were treated with both 12 mg PG and placebo were available for analysis. Treatment with 12 mg PG, 40 mg Omeprazole and sodium bicarbonate resulted in 40.1±2% (mean±SEM) percent of time of gastric pH above 4, whereas treatment with PG placebo (40 mg Omeprazole and sodium bicarbonate only) resulted in 31.6±2% percent of time of gastric pH above 4. Using ANOVA (mixed model) the p value was 0.019. When night time was analyzed seperately, the treatment with 12 mg PG, 40 mg Omeprazole and sodium bicarbonate resulted in 34.3±2.6% percent of time of gastric pH above 4 whereas 40 mg Omeprazole and sodium bicarbonate resulted in 24.9±2.6% percent of time of gastric pH above 4 (p=0.041).

Safety Results:

The primary safety endpoint of the percent of post-dose daytime and nocturnal time of gastric pH below 1.5 was not significantly different between the placebo, 4 mg PG or 12 mg PG treatments. No clinical adverse events were reported during the 24 hour pH monitoring. There were no significant differences in heart rate or blood pressure between treatment arms (12 mg PG versus placebo). There were no significant differences in the laboratory results in the treatment arms. Preliminary results of the pharmacokinetic analysis for PG blood levels indicated that no PG could be detected in the peripheral blood circulation (lower limit of detection 1 ng/ml).

Conclusions:

The single oral administration of PG (4 mg and 12 mg) was not associated with an increase in physical or clinical adverse events or laboratory test abnormalities. The efficacy results suggest that oral PG may enhance the effect of omeprazole in suppressing the secretion of gastric acid. Importantly, these results highlight the importance of the synchronization between the PK/PD effects of both components in the design of the final formulation. The post-hoc analysis demonstrated advantage for the PG-omeprazole-bicarbonate combination as compared to the omeprazole-bicarbonate only, supporting the concept of pre-stimulation of gastric parietal cells as a mean to enhance the effect of PPIs.

F: A Kit of PPI with Adjustable Amount of Buffer

### Example 19

### The Effect of Increasing Amount of Sodium Bicarbonate on the pH of Stimulated Gastric Fluid

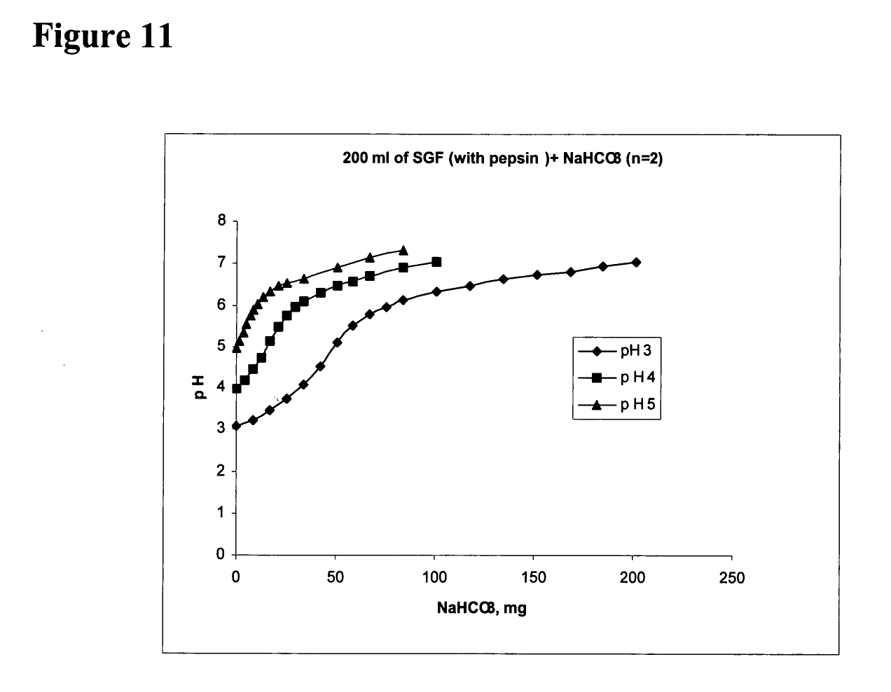

The effect of increasing amount of sodium bicarbonate on the pH of stimulated gastric fluid (SGF) was determined in vitro by adding escalated amounts of sodium bicarbonate to artificial gastric fluid in different pre-determined pH. Stimulated gastric fluid was prepared in accordance with U.S. Pharmacopoeia (USP) 2000 Ed., P. 235. For preparing 200 ml of gastric fluid, 0.4 g of NaCl and 0.64 g of Pepsin were dissolved in 16 ml 1M HCl and 184 ml of water (final concentration of HCl is 80 mM). Three solutions of 200 ml Gastric fluid (containing Pepsin) were prepared at pH-3, pH-4 and pH-5 by adding different amounts of HCL. The Gastric Fluid was titrated with increasing amounts of NaHCO3. As shown in FIG. 11, when the initial pH of the SGF is 4.0, less than 100 mg of sodium bicarbonate were needed to raise the pH of modified gastric fluid (initial pH 3, 4 or 5 mimicking chronic PPI treatment conditions) to pH around 6.

Table 3 below summarizes the results obtained under conditions that simulate chronic PPI administration and continuance acid secretion (initial gastric pH of 4; acid secretion rate of approximately 3 mmol HCl/hour). As revealed from this table, 336 mg of sodium bicarbonate were sufficient to maintain the pH at about 5.7 for at least one hour with simulation of 3 mmol HCL secretion per hour. This implies that this amount of sodium bicarbonate may preserve the pH in the stomach above 5.7 for at least one hour.

TABLE 3
Titration of water solutions starting from pH 4, containing
different amounts of NaHCO₃ with HCl

| NaHCO3, mg | 252 | 336 | 420 | 600 | 714 |
|---|---|---|---|---|---|
| Initial pH | 4.02 | 4.02 | 3.95 | 4.01 | 4.05 |
| mM HCl/hour | pH | | | | |
| 0 | 8.9 | 8.92 | 8.92 | 8.9 | 8.9 |
| 3 | 4.55 | 5.77 | 6.10 | 6.44 | 6.55 |
| 6 | 1.56 | 1.73 | 2.06 | 5.6 | 5.88 |
| 9 | | | | 1.84 | 2.39 |

### Example 20

### Composition with Adjustable Amount of Buffer

This example describes a kit for oral delivery containing a start dose (first six days of treatment) and a continuance dose. Both doses comprise omeprazole as a PPI, sodium bicarbonate as a buffering agent and Pentagastrin as a parietal cell activator.

Initial Dose for the First Six Days:

Capsule I/effervescent tablet: 1300 mg NaHCO3

Capsule II: 40 mg Pentagastrin
80 mg omeprazole
300 mg NaHCO3

Continuance Dose for Consequent Use:

Single capsule: 40 mg Pentagastrin
80 mg omeprazole
300 mg NaHCO3

It will be appreciated by a person skilled in the art that the present invention is not limited by what has been particularly shown and described hereinabove. Rather, the scope of the invention is defined by the claims that follow.

1. An oral pharmaceutical composition comprising as active ingredients a pharmaceutically effective amount of: (i) a peptide comprising the amino acid sequence of SEQ ID NO:1, which activates parietal cells; (ii) an irreversible gastric H⁺/K⁺-ATPase proton pump inhibitor (PPI); and (iii) at least one preservation agent that preserves the availability of the peptide in gastric fluids.

2. The oral composition of claim 1, wherein the peptide is pentagastrin (PG) having the amino acid sequence of SEQ ID NO:2 or a synthetic analog thereof.

3. The oral composition of claim 2, wherein the preservation agent is one or more pH regulating agents, wherein the amount of the pH regulating agent is sufficient to preserve the availability of PG in the stomach so that the biological activity of PG is maintained.

4. The oral composition of claim 3, wherein the one or more pH regulating agents are selected from the group consisting of: sodium bicarbonate, potassium bicarbonate, magnesium oxide, magnesium hydroxide, magnesium carbonate, magnesium lactate, magnesium glucomate, aluminum hydroxide, sodium carbonate, potassium carbonate, phosphate carbonate, citrate carbonate, di-sodium carbonate, disodium hydrogen phosphate, aluminum glycinate, calcium hydroxide, calcium lactate, calcium carbonate and calcium bicarbonate, or a mixture thereof.

5. The oral composition of claim 3, wherein the oral composition is formulated in a single unit dosage form and the pH regulating agent is in an amount of at least 300 mg.

6. The oral composition of claim 2, wherein the peptide is in an amount sufficient to locally activate parietal cells located in the gastric lumen.

7. The oral composition of claim 2, wherein the active ingredients are formulated in a single unit dosage form.

8. The oral composition of claim 7, wherein the amount of PG is between 2 to 60 mg.

9. The oral composition of claim 7, wherein the single unit dosage form is a double-layered tablet, a press-coat tablet, a multi-particulate capsule, an effervescent tablet, a suspension tablet, solution, or suspension comprising PPI beads, PG beads and at least one pH regulating agent.

10. The oral composition of claim 9, wherein the pH regulating agent is in an amount sufficient to preserve-the availability of PG in the stomach so that the biological activity of PG is maintained.

11. The oral composition of claim 10, wherein the PPI beads and the PG beads are coated with enteric-coating or with time-dependent release polymers, wherein the release of the PPI from the PPI beads precedes the release of PG from the PG beads.

12. The oral composition of claim 11, wherein the time-dependent release polymers comprise at least one polymer capable of swelling in aqueous environment.

13. The oral composition of claim 12, wherein at least one polymer is selected from the group consisting of: a synthetic polymer and cellulose-based polymer, or substituted derivative thereof.

14. The oral composition of claim 11, wherein the PG beads further comprise at least one carbonate salt capable of reacting with gastric acid to form carbon dioxide which is entrapped within the PG beads, thereby inducing the buoyancy of said PG beads over the gastric juice.

15. The oral composition of claim 14, wherein the carbonate salts are sodium bicarbonate or calcium carbonate.

16. The oral composition of claim 10, comprising non-coated PPI beads, PG beads and at least one pH regulating agents, wherein the release of PG from the PG beads is delayed relative to the release of the PPI from the PPI beads.

17. The oral composition of claim 16, wherein the at least one pH regulating agents is selected from the group consisting of: calcium carbonate, sodium or potassium bicarbonate, magnesium oxide, hydroxide or carbonate, magnesium lactate, magnesium gluconate, aluminum hydroxide, aluminium, calcium, sodium or potassium carbonate, phosphate or citrate, di-sodium carbonate, disodium hydrogen phosphate, a mixture of aluminum glycinate and a buffer, calcium hydroxide, calcium lactate, calcium carbonate and calcium bicarbonate.

18. The oral composition of claim 3, wherein the pH regulating agent is in an amount sufficient to raise the pH in the stomach to a pH of at least 4.5.

19. The oral composition of claim 1, wherein the PPI is selected from the group consisting of: rabeprazole, omeprazole, isomeprazole, lansoprazole, pantoprazole, leminoprazole, single enantiomers thereof, alkaline salts thereof and mixtures thereof.

20. The oral composition of claim 1, wherein the composition further comprising a pepsin inhibitor, a mucolytic agent or an antibiotic effective against bacteria residing in the stomach.

21. The oral composition of claim 2, wherein the peptide is the N-protected derivative of PG selected from the group consisting of: methoxymethyl (MOM), β-methoxyethoxymethyl (MEM), trialkylsilyl, triphenylmethyl (trityl), TIPSO, tert-butoxycarbonyl (t-BOC), ethoxyethyl (EE), F-MOC, and TROC.

22 The oral composition of claim 18, wherein the pH regulating agent is in amount sufficient to raise the pH of the stomach to a pH of at least 5.5.

23. The oral pharmaceutical composition of claim 1, further comprising a gastric acid stimulant selected from the group consisting of: a dicarboxylic acid molecule, tricarboxylic acid molecule, or a combination thereof.

24. The oral composition of claim 23, wherein the dicarboxylic acid or tricarboxylic acid molecule is selected from the group consisting of: succinic acid, maleic acid, citric acid and fumaric acid.

25. An oral pharmaceutical kit comprising as active ingredients a pharmaceutically effective amount of: (i) a peptide comprising the amino acid sequence of SEQ ID NO:1; (ii) an irreversible gastric H⁺/K⁺-ATPase proton pump inhibitor (PPI); and (iii) at least one agent that preserves the availability of the peptide in the gastric fluids for at least 20 to 30 minutes, wherein the active ingredients are formulated in separate unit dosage forms.

26. A method of treating or preventing a disorder in a subject in which suppression of gastric acid secretion is required, comprising administering to a subject in need of such treatment a therapeutically effective amount of a composition according to claim 1.

27. The method of claim 26, wherein the disorder is selected from the group consisting of: reflux esophagitis, gastritis, duodenitis, gastric ulcer, duodenal ulcer, pathologies associated with nonsteroidal anti-inflammatory drugs (NSAID), non-ulcer Dyspepsia, gastro-esophageal reflux disease, gastrinomas, acute upper gastrointestinal bleeding, stress ulceration, Helicobacter pylori infections, Zollinger-Ellison syndrome (ZES), Werner's syndrome, and systemic mastocytosis.

28. A pharmaceutical kit for oral PPI treatment comprising:
(a) an initial dose for the early stage of PPI treatment comprising a pharmaceutically effective amounts of a PPI combined with a peptide comprising the amino acid sequence of SEQ ID NO:1 and one or more pH regulating agents, wherein the pH regulating agents are in an amount sufficient to raise the pH in the stomach to a pH of at least 4.5, and
(b) a continuance dose for the subsequent stage of PPI treatment comprising the effective amounts of the PPI and the peptide and one or more pH regulating agents in an amount less than the initial dose, but sufficient to raise the pH in the stomach to a pH of at least 4.5.

29. A method of reducing gastric acid secretion in a mammal, the method comprising orally administering to the mammal an effective amount of a pH regulating agent and a peptide comprising SEQ ID NO:1 in conjunction with an effective amount of a proton pump inhibitor (PPI), wherein the pH regulating agent is administered in an amount sufficient to raise the pH in the stomach to a pH greater than 4.5 for a sufficient time that the peptide enhances the inhibitory activity of PPI, thereby reducing the gastric acid secretion in the mammal.
